SEQUENCE LISTING (13 sequences)

SEQ ID NO: 1 - DNA, 2247 bp, Unknown
  Description of Unknown STAT4-alpha isoform sequence
atgtctcagtggaatcaagtccaacagttagaaatcaagtttttggagcaggtggatcaattctatgatgacaactttcccatggaaattcggcatctgttggcccaatggattgaaaatcaagactgggaggcagcttctaacaatgaaaccatggcaacgattcttcttcaaaacttgttaatacaactggatgaacagttaggtcgtgtttccaaagagaaaaacctactcttgatacacaatctaaaaagaattaggaaggtccttcagggaaaatttcatggaaatccaatgcatgtagctgtggttatttcaaactgtttaagggaagagaggagaatattggctgcagccaacatgcctgtccaggggcctctagagaaatccttacaaagttcttcagtttcagaaagacagaggaatgtggagcacaaagtggctgccattaaaaacagtgtgcagatgacagaacaagataccaaatacttagaagatctgcaagacgaatttgactacaggtataaaacaattcagacaatggatcagagtgacaagaatagtgccatggtgaatcaggaagttttgacactgcaggaaatgcttaacagcctcgatttcaagagaaaggaggctctcagtaaaatgacccaaatcatccatgagacagacctgttaatgaacaccatgctcatagaagagctgcaagactggaagcggcggcagcaaatcgcctgcatcgggggtccactccacaatgggctcgaccagcttcagaactgctttacactattggcagaaagtcttttccaactgagaaggcaattggagaaactagaggagcaatctaccaaaatgacatatgaaggtgatcccattccaatgcaaagaactcacatgctagaaagagtcaccttcttgatctacaaccttttcaagaactcatttgtggttgagcgacagccatgtatgccaacccaccctcagaggccgttggtacttaaaaccctaattcagttcactgtaaaactaaggctactaataaaattgccagaactaaactatcaggtaaaggttaaggcatcaattgacaagaatgtttcaactctaagcaaccgaagatttgtactttgtggaactaatgtcaaagccatgtctattgaagaatcttccaatgggagtctctcagtagaatttcgacatttgcaaccaaaggaaatgaagtccagtgctggaggtaaaggaaatgagggctgtcacatggtgactgaagaacttcattccataacgtttgaaacacagatctgcctctatggcctgaccatagatttggagaccagctcattgcctgtggtgatgatttccaatgtcagtcagttacctaatgcttgggcatccatcatttggtacaacgtgtcaaccaacgattcccagaacttggttttctttaataatcctccacctgccacattgagtcaactactggaggtgatgagctggcagttttcatcgtacgttggtcgtggtcttaactcagatcaactccatatgctggcagagaagcttacagtccaatctagctacagtgatggtcacctcacctgggccaagttctgcaaggaacatttacctggtaaatcatttaccttttggacatggcttgaagcaatattggatctaattaagaaacacattcttcccctttggattgatgggtatgtcatgggctttgttagcaaagagaaggaacggctgttgctaaaggataaaatgcctggcacctttttattaagattcagtgaaagccatctcggaggaataactttcacctgggtggaccattctgaaagtggggaagtgagattccactctgtagaaccctacaataaaggccggttgtctgctctgccattcgctgacatcctgcgagactacaaagttattatggctgaaaacattcctgaaaaccctctgaagtacctatatcctgacattcccaaagacaaagccttcggtaaacactacagctctcagccttgcgaagtttcaagaccaacagaaaggggtgacaaaggttatgttccttctgtttttatccccatctcaacaatccgaagtgattcaacagagccacattctccatcagaccttcttcccatgtctccaagtgtgtatgcggtgttgagagaaaacctgagtcccacaacaattgaaactgcaatgaagtctccttattctgctgaatga

SEQ ID NO: 2 - DNA, 2616 bp, Unknown
  Description of Unknown STAT4-beta sequence
atgtctcagtggaatcaagtccaacagttagaaatcaagtttttggagcaggtggatcaattctatgatgacaactttcccatggaaattcggcatctgttggcccaatggattgaaaatcaagactgggaggcagcttctaacaatgaaaccatggcaacgattcttcttcaaaacttgttaatacaactggatgaacagttaggtcgtgtttccaaagagaaaaacctactcttgatacacaatctaaaaagaattaggaaggtccttcagggaaaatttcatggaaatccaatgcatgtagctgtggttatttcaaactgtttaagggaagagaggagaatattggctgcagccaacatgcctgtccaggggcctctagagaaatccttacaaagttcttcagtttcagaaagacagaggaatgtggagcacaaagtggctgccattaaaaacagtgtgcagatgacagaacaagataccaaatacttagaagatctgcaagacgaatttgactacaggtataaaacaattcagacaatggatcagagtgacaagaatagtgccatggtgaatcaggaagttttgacactgcaggaaatgcttaacagcctcgatttcaagagaaaggaggctctcagtaaaatgacccaaatcatccatgagacagacctgttaatgaacaccatgctcatagaagagctgcaagactggaagcggcggcagcaaatcgcctgcatcgggggtccactccacaatgggctcgaccagcttcagaactgctttacactattggcagaaagtcttttccaactgagaaggcaattggagaaactagaggagcaatctaccaaaatgacatatgaaggtgatcccattccaatgcaaagaactcacatgctagaaagagtcaccttcttgatctacaaccttttcaagaactcatttgtggttgagcgacagccatgtatgccaacccaccctcagaggccgttggtacttaaaaccctaattcagttcactgtaaaactaaggctactaataaaattgccagaactaaactatcaggtaaaggttaaggcatcaattgacaagaatgtttcaactctaagcaaccgaagatttgtactttgtggaactaatgtcaaagccatgtctattgaagaatcttccaatgggagtctctcagtagaatttcgacatttgcaaccaaaggaaatgaagtccagtgctggaggtaaaggaaatgagggctgtcacatggtgactgaagaacttcattccataacgtttgaaacacagatctgcctctatggcctgaccatagatttggagaccagctcattgcctgtggtgatgatttccaatgtcagtcagttacctaatgcttgggcatccatcatttggtacaacgtgtcaaccaacgattcccagaacttggttttctttaataatcctccacctgccacattgagtcaactactggaggtgatgagctggcagttttcatcgtacgttggtcgtggtcttaactcagatcaactccatatgctggcagagaagcttacagtccaatctagctacagtgatggtcacctcacctgggccaagttctgcaaggaacatttacctggtaaatcatttaccttttggacatggcttgaagcaatattggatctaattaagaaacacattcttcccctttggattgatgggtatgtcatgggctttgttagcaaagagaaggaacggctgttgctaaaggataaaatgcctggcacctttttattaagattcagtgaaagccatctcggaggaataactttcacctgggtggaccattctgaaagtggggaagtgagattccactctgtagaaccctacaataaaggccggttgtctgctctgccattcgctgacatcctgcgagactacaaagttattatggctgaaaacattcctgaaaaccctctgaagtacctatatcctgacattcccaaagacaaagccttcggtaaacactacagctctcagccttgcgaagtttcaagaccaacagaaaggggtgacaaaggttatgttccttctgtttttatccccatctcaacaatgtgagtaatgttagtcacatgtgaaatatttttataaaaagctttcctataggagatttaaaggtagagcagagtacacataactgagaacaaagcattgtaatgtgcaatgtcccatttcctttaatacataaggctagccttcagggcacacttaccacaatctattgtgcctaaaattataaaattccccttttatatgccatatatgccacagtaagttgagtgttctgatatgaaatgatgaattagataactcaatgtcacaaatagatgaagccctagaaatgagttcctgacatagtaagtcaccgtgaactattattattttttaatccttgtccatattgaccttgttatctctttaagccgaagtgattcaacagagccacattctccatcagaccttcttcccatgtctccaagtgtgtatgcggtgttgagagaaaacctgagtcccacaacaattgaaactgcaatgaagtctccttattctgctgaatga

SEQ ID NO: 3 - DNA, 369 bp, Unknown
  Description of Unknown STAT4-beta specific exon sequence
gtgagtaatgttagtcacatgtgaaatatttttataaaaagctttcctataggagatttaaaggtagagcagagtacacataactgagaacaaagcattgtaatgtgcaatgtcccatttcctttaatacataaggctagccttcagggcacacttaccacaatctattgtgcctaaaattataaaattccccttttatatgccatatatgccacagtaagttgagtgttctgatatgaaatgatgaattagataactcaatgtcacaaatagatgaagccctagaaatgagttcctgacatagtaagtcaccgtgaactattattattttttaatccttgtccatattgaccttgttatctctttaag

SEQ ID NO: 4 - protein, 748 aa, Unknown
  Description of Unknown STAT4-alpha sequence
MSQWNQVQQLEIKFLEQVDQFYDDNFPMEIRHLLAQWIENQDWEAASNNETMATILLQNLLIQLDEQLGRVSKEKNLLLIHNLKRIRKVLQGKFHGNPMHVAVVISNCLREERRILAAANMPVQGPLEKSLQSSSVSERQRNVEHKVAAIKNSVQMTEQDTKYLEDLQDEFDYRYKTIQTMDQSDKNSAMVNQEVLTLQEMLNSLDFKRKEALSKMTQIIHETDLLMNTMLIEELQDWKRRQQIACIGGPLHNGLDQLQNCFTLLAESLFQLRRQLEKLEEQSTKMTYEGDPIPMQRTHMLERVTFLIYNLFKNSFVVERQPCMPTHPQRPLVLKTLIQFTVKLRLLIKLPELNYQVKVKASIDKNVSTLSNRRFVLCGTNVKAMSIEESSNGSLSVEFRHLQPKEMKSSAGGKGNEGCHMVTEELHSITFETQICLYGLTIDLETSSLPVVMISNVSQLPNAWASIIWYNVSTNDSQNLVFFNNPPPATLSQLLEVMSWQFSSYVGRGLNSDQLHMLAEKLTVQSSYSDGHLTWAKFCKEHLPGKSFTFWTWLEAILDLIKKHILPLWIDGYVMGFVSKEKERLLLKDKMPGTFLLRFSESHLGGITFTWVDHSESGEVRFHSVEPYNKGRLSALPFADILRDYKVIMAENIPENPLKYLYPDIPKDKAFGKHYSSQPCEVSRPTERGDKGYVPSVFIPISTIRSDSTEPHSPSDLLPMSPSVYAVLRENLSPTTIETAMKSPYSAE

SEQ ID NO: 5 - protein, 704 aa, Unknown
  Description of Unknown STAT4-beta sequence
MSQWNQVQQLEIKFLEQVDQFYDDNFPMEIRHLLAQWIENQDWEAASNNETMATILLQNLLIQLDEQLGRVSKEKNLLLIHNLKRIRKVLQGKFHGNPMHVAVVISNCLREERRILAAANMPVQGPLEKSLQSSSVSERQRNVEHKVAAIKNSVQMTEQDTKYLEDLQDEFDYRYKTIQTMDQSDKNSAMVNQEVLTLQEMLNSLDFKRKEALSKMTQIIHETDLLMNTMLIEELQDWKRRQQIACIGGPLHNGLDQLQNCFTLLAESLFQLRRQLEKLEEQSTKMTYEGDPIPMQRTHMLERVTFLIYNLFKNSFVVERQPCMPTHPQRPLVLKTLIQFTVKLRLLIKLPELNYQVKVKASIDKNVSTLSNRRFVLCGTNVKAMSIEESSNGSLSVEFRHLQPKEMKSSAGGKGNEGCHMVTEELHSITFETQICLYGLTIDLETSSLPVVMISNVSQLPNAWASIIWYNVSTNDSQNLVFFNNPPPATLSQLLEVMSWQFSSYVGRGLNSDQLHMLAEKLTVQSSYSDGHLTWAKFCKEHLPGKSFTFWTWLEAILDLIKKHILPLWIDGYVMGFVSKEKERLLLKDKMPGTFLLRFSESHLGGITFTWVDHSESGEVRFHSVEPYNKGRLSALPFADILRDYKVIMAENIPENPLKYLYPDIPKDKAFGKHYSSQPCEVSRPTERGDKGYVPSVFIPISTM

SEQ ID NO: 6 - DNA, 21 bp, Artificial Sequence
  Description of Artificial Sequence Synthetic primer
tatcctgacattcccaaagac

SEQ ID NO: 7 - DNA, 21 bp, Artificial Sequence
  Description of Artificial Sequence Synthetic primer
ctctcaacaccgcatacacac

SEQ ID NO: 8 - DNA, 21 bp, Artificial Sequence
  Description of Artificial Sequence Synthetic primer
gacttactatgtcaggaactc

SEQ ID NO: 9 - protein, 21 aa, Mus sp.
MEVGWYRSPFSRVVHLYRNGK

SEQ ID NO: 10 - DNA, 171 bp, Unknown
  Description of Unknown STAT4-alpha sequence
tatgttccttctgtttttatccccatctcaacaatccgaagtgattcaacagagccacattctccatcagaccttcttcccatgtctccaagtgtgtatgcggtgttgagagaaaacctgagtcccacaacaattgaaactgcaatgaagtctccttattctgctgaatga

SEQ ID NO: 11 - protein, 56 aa, Unknown
  Description of Unknown STAT4-alpha sequence
YVPSVFIPISTIRSDSTEPHSPSDLLPMSPSVYAVLRENLSPTTIETAMKSPYSAE

SEQ ID NO: 12 - protein, 12 aa, Unknown
  Description of Unknown STAT4-beta sequence
YVPSVFIPISTM

SEQ ID NO: 13 - DNA, 540 bp, Unknown
  Description of Unknown STAT4-beta sequence
tatgttccttctgtttttatccccatctcaacaatgtgagtaatgttagtcacatgtgaaatatttttataaaaagctttcctataggagatttaaaggtagagcagagtacacataactgagaacaaagcattgtaatgtgcaatgtcccatttcctttaatacataaggctagccttcagggcacacttaccacaatctattgtgcctaaaattataaaattccccttttatatgccatatatgccacagtaagttgagtgttctgatatgaaatgatgaattagataactcaatgtcacaaatagatgaagccctagaaatgagttcctgacatagtaagtcaccgtgaactattattattttttaatccttgtccatattgaccttgttatctctttaagccgaagtgattcaacagagccacattctccatcagaccttcttcccatgtctccaagtgtgtatgcggtgttgagagaaaacctgagtcccacaacaattgaaactgcaatgaagtctccttattctgctgaatga

(19) United States
(12) Patent Application Publication
(10) Pub. No.: US 2011/0200600 A1
(43) Pub. Date: Aug. 18, 2011

(54) DIAGNOSIS AND PROGNOSIS OF IMMUNE DISORDERS USING STAT4 EXPRESSION

(75) Inventor: Mark H. Kaplan, Fishers, IN (US)

(73) Assignee: Indiana University Research and Technology Corporation

(21) Appl. No.: 13/063,094

(22) PCT Filed: Sep. 9, 2009
(86) PCT No.: PCT/US09/56332

§ 371 Date: Apr. 29, 2011

Related U.S. Application Data
(60) Provisional application No. 61/095,684, filed on Sep. 10, 2008.

(51) Int. Cl. A61K 39/395; C12Q 1/68; G01N 33/68; G01N 33/53; A61K 38/02; C07H 21/00; C12Q 1/02; A61P 29/00; A61P 1/00; A61P 37/06; H01J 49/26

(52) U.S. Cl. 424/134.1; 435/6.1; 435/6.11; 435/6.12; 435/7.1; 436/501; 424/133.1; 424/142.1; 424/145.1; 514/1.1; 514/1.4; 536/24.31; 435/29; 435/6.13; 250/282

(57) ABSTRACT

Methods and compositions that determine the expression levels of Stat4α and Stat4β isoforms for therapeutic efficacy of anti-inflammatory treatments, assessing an individual's risk for developing inflammatory diseases including Crohn's disease, ulcerative colitis, rheumatoid arthritis, and multiple sclerosis are disclosed.

This application claims priority to U.S. Ser. No. 61/095,684 filed Sep. 10, 2008, the contents of which is incorporated by reference in its entirety.

The United States Government has rights in this invention pursuant to funding under National Institutes of Health grant number AI045515.

### BACKGROUND

The present disclosure relates to determining therapeutic efficacy of anti-TNF therapies for treating inflammatory diseases including inflammatory bowel diseases such as Crohn's and ulcerative colitis.

Signal Transducer and Activator of Transcription (STAT) proteins are a family of factors implicated in a variety of biological processes. STAT proteins exist as latent monomers within the cytoplasm of cells. Following interaction of a cytokine/growth factor with a cell surface receptor, STATs are recruited to the receptor through specific interactions between the STAT SH2 domain and receptor phosphotyrosines. The STAT then is tyrosine phosphorylated and can form homodimers through reciprocal interactions between phosphotyrosines and SH2 domains of two STAT monomers. The dimers then move to the nucleus, bind DNA and modulate gene transcription. This mechanism provides a direct link between cell surface cytokine/growth factor stimulation and gene activation in the nucleus. Stat4 is a member of the STAT family of proteins.

Stat4 was first cloned by cross hybridization with other cloned STAT proteins. It is the only STAT protein that shows tissue-restricted expression, with mRNA found mainly in lymphoid and myeloid tissues. The Stat4 monomer is a 90 kDa protein with an N-terminal domain important for the interaction of multiple Stat4 dimers, a coiled coil interaction domain, a DNA binding domain, an SH2 domain and a tyrosine important for dimerization. Stat4 contains a C-terminal transactivation domain (TAD) and phosphorylation of a serine residue within this domain affects transactivation.

Stat proteins are expressed as multiple isoforms; alpha forms that are full length and beta forms that lack the C-terminal transactivation domain of the alpha form and rather have a novel Cterminal domain resulting from the lack of splicing of the last exon. Although the isoform phenomenon is well documented, the biological role of these isoforms is not entirely clear. For Stat1 and Stat5, the beta isoforms are dominant negatives. The functions of Stat3 are more context dependent, where the beta isoform may interfere with transcription of some genes but activate others. Indeed, Stat3β can mediate some aspects of liver inflammation and rescue the embryonic lethality of Stat3-deficiency. Stat4 is also expressed as two isoforms, a full length form termed Stat4α and a β isoform that lacks the TAD, termed Stat4β (Hoey et al., (2003) Distinct requirements for the naturally occurring splice forms Stat4α and Stat4β in IL-12 responses, The EMBO Journal, vol. 22:16 pp. 4237-4248). Each Stat4 isoform is able to mediate Th1 differentiation in vitro. However, the role of Stat4 isoforms in the pathogenesis of organ-specific autoimmune diseases in vivo has only recently been examined.

IL-12 was a hallmark cytokine demonstrated to stimulate the activation of Stat4. Stat4 is also activated by IFNγ, though differently in human and mouse cells. Stat4 activation by IFNγ may be important in anti-viral responses. IL-23 has also been shown to activate Stat4, though whether Stat4 mediates any IL-23-stimulated biological functions is still unclear. The cytokine binds specifically to two non-covalently linked receptor chains expressed on NK, activated T and B cells. The chains are termed IL-12Rβ1 and IL-12Rβ2 since both chains have homology to β chains of the gp130 family of receptors. The β2 chain is tyrosine phosphorylated and is responsible for recruitment and activation of Stat4. The biological effects of IL-12 include induction of IFNγ expression in NK and activated T cells, increasing cytotoxic responses in both T and NK cells, inducing proliferation of activated T cells and stimulating the development of fully functional Th1 cells. IL-12 has also been implicated in many inflammatory diseases.

IL-23 is a heterodimeric cytokine composed of the IL-12 p40 chain disulfide linked to a novel p19 protein. IL-23 activates similar Janus kinases to IL-12 as well as activating of Stat1, Stat3 and Stat4. In vivo, IL-23 promotes inflammation and is critical for the development of experimental autoimmune encephalomyelitis, supporting a potentially important role for IL-23 in disease.

In an analysis of mice deficient in Stat4, it was shown that Stat4 is required for all known IL-12 biological functions, including the induction of IFN-γ and the promotion of Th1 differentiation. Despite the deficiencies in IL-12 signaled function, there were no obvious defects in the mature myeloid cell compartment. Furthermore, with the exception of the loss of IL-12 responses, the immune system appeared normal. This demonstrates the exquisite specificity of Stat4 function. It also demonstrates that while other STAT proteins may be activated by IL-12, they are not sufficient to achieve any of the known IL-12 responses.

The phenotype of disease in Stat4-deficient mice demonstrates the requirement for Stat4 in Type 1 immunity. Stat4-deficient mice are susceptible to infection with Trypanosoma cruzi, Toxoplasma gondii, Leishmania major, Leishmania mexicana, Mycobacterium tuberculosis, and have decreased DTH responses. In contrast, Stat4-deficient mice are refractory to the induction of inflammatory conditions including colitis, arthritis, diabetes, adhesion formation, myocarditis, cardiac allograft vasculopathy, endotoxemia, renal and hepatic ischemia-reperfusion injury and experimental autoimmune encephalitis. T cell memory responses in Stat4-deficient mice generate little IFN-γ. Thus, the phenotype of the Stat4-deficient model is that of a mouse with greatly impaired Th1 responses in vivo.

Chronic inflammatory bowel disease (IBD) that affects the intestine (Crohn's Disease, CD) or colon (ulcerative colitis) is increasing in incidence and while mortality is low, symptoms may be debilitating. The need for new treatments and diagnosis tools is great. In mouse models, Stat4 is required for the development of IBD and increased expression of Stat4 results in IBD. It was thought for many years that Th1 cells were critical in the development of Crohn's disease. However, experiments have demonstrated the involvement of Th17 cells and IL-23 in the development of CD. Importantly, Stat4 is a critical factor in the development of Th1 and Th17 cells. In patient samples, constitutively active Stat4 has been observed in intestinal T cells and inflammed mucosal tissue samples from patients with CD. Thus, factors that regulate Stat4 activity are of great interest in understanding disease pathogenesis and may aid in further treatment of disease. To date, the most effective therapy has been aminosalicylates, sulfasalazine, corticosteroids and anti-TNF-α therapy, all of which either limit the production or activity of proinflammatory cytokines secreted by the leukocytes

Multiple Sclerosis (MS) is an inflammatory demyelinating disease of the central nervous system (CNS) that afflicts more than one million people worldwide. The disease usually begins in young adults and affects women more frequently than men. About 30% of MS patients develop clinical paralysis and become wheel chair-bound for the rest of their lives. There is currently no medical treatment available that can cure MS. The destruction of the oligodendrocyte myelin sheath and axonal loss in the CNS are the pathological hall-marks of MS. Although the etiology of MS remains unknown, it is generally viewed as an organ-specific autoimmune disease, mediated by myelin-reactive T cells in the CNS. Activation of immune cells, secretion of inflammatory cytokines and differentiation of encephalitogenic T cells are key processes associated with the pathogenesis of MS. Experimental allergic encephalomyelitis (EAE) is a CD4+ Th1/Th17 cell-mediated inflammatory demyelinating autoimmune disease of the CNS. EAE can be induced in susceptible animals by immunization with whole brain homogenate and purified neural antigens such as myelin basic protein (MBP), proteolipid protein (PLP) and myelin oligodendrocyte glycoprotein (MOG) or adoptive transfer of neural antigen specific T cells. The clinical and pathological features of EAE show close similarity to human MS and therefore it has commonly been used as an animal model to study the mechanisms of MS pathogenesis and to test the efficacy of potential therapeutic agents for the treatment of MS.

In the present disclosure, use of the expression ratio of Stat4α/Stat4β in evaluating inflammatory inflammatory diseases was analyzed.

### SUMMARY

A method of predicting the likelihood of successful anti-inflammatory therapy for a patient suffering from an inflammatory disease includes: (a) determining the expression level of Stat4α and Stat4β isoforms in a biological sample from the patient; and (b) predicting the likelihood of successful anti-TNF therapy for a patient suffering from an inflammatory disease by correlating the relative expression levels of Stat4α and Stat4β isoforms in the patient to a control sample or a reference value.

A method of assessing a patient's risk for developing an inflammatory disease or an inflammatory response includes: (a) quantifying the expression level of Stat4α and Stat4β isoforms in a biological sample from the patient; and (b) determining that the patient's risk for the inflammatory disease or the inflammatory response is higher if the patient exhibits a higher Stat4β:Stat4α ratio as compared to a control.

A method of predicting disease severity in a patient's suspected of suffering from an inflammatory disease includes (a) obtaining the expression level of Stat4α and Stat4β isoforms in a biological sample from the patient; and (b) determining that the disease severity for the patient suffering from the inflammatory disease is higher if the patient exhibits a higher Stat4β:Stat4α ratio as compared to a control.

A method of preventing or minimizing excessive inflammatory response in an immuno compromised patient includes: (a) determining if the patient exhibits higher risk for the inflammatory response based on the patient's Stat4β:Stat4α expression level ratio in a biological sample as compared to a control; and (b) administering an anti-inflammatory therapy to minimize the excessive inflammatory response.

A suitable biological sample is blood. The expression level of Stat4α and Stat4β may be determined by analyzing the expression peripheral blood mononuclear cells (PBMC).

A suitable inflammatory disease or response is selected from the group consisting of Crohn's disease, ulcerative colitis, rheumatoid arthritis, juvenile rheumatoid arthritis, psoriatic arthritis, ankylosing spondylitis, lupus, asthma, psoriasis, type I diabetes, carditis, chronic obstructive pulmonary disease (COPD, inflammatory bowel disease (IBD), and multiple sclerosis (MS).

Suitable ratio of Stat4β/Stat4α ranges from about to 0.1 to 60.0 or higher. Other ratios (e.g., less than about 0.1 and greater than about 60) are also suitable depending upon the inflammatory condition.

The expression level of Stat4α and Stat4β may be determined by any technique including but not limited to PCR, quantitative PCR or real-time PCR, semi-quantitative PCR, probe-hybridization, and antibody-based quantitation.

Suitable clinicopathological data, if necessary, may be selected from patient age, previous personal and/or familial history of inflammatory diseases, previous personal and/or familial history of response to anti-inflammatory therapy, and presence of one or more single nucleotide polymorphisms (SNPs) associated with the Stat4 isoforms.

Suitable anti-inflammatory therapy includes but not limited to infliximab, adalimumab, certolizumab pegol, afelimomab, golimumab, etanercept, abatacept, and anakinra.

The expression levels of Stat4α and Stat4β may be useful for a clinical detection of disease, disease diagnosis, disease prognosis, or treatment outcome or a combination of any two, three or four of these actions.

In an embodiment, an excessive inflammatory response is associated with sepsis. In an embodiment, the immune compromised patient is treated with an immuno suppressive agent.

A diagnostic kit to predict the response to anti-inflammatory therapy includes oligonucleotide primers to specifically detect and quantify the expression levels of Stat4α and Stat4β isoforms.

Suitable oligonucleotide primers include for example 5′-TAT CCT GAC ATT CCC AAA GAC-3′ (SEQ ID NO: 6), 5′-CTC TCA ACA CCG CAT ACA CAC-3′ (SEQ ID NO: 7), and 5′ GAC TTA CTA TGT CAG GAA CTC-3′ (SEQ ID NO: 8). Other oligo nucleotide primers can be readily designed based on the sequences disclosed herein and as shown in FIG. 15.

A nucleic acid probe or a primers includes a contiguous region of about 15 nucleotides of SEQ ID NO: 5, wherein the probe is capable of selectively binding to the Stat4β-specific exon. In an embodiment, a probe includes a reverse complementary strand capable of selectively binding to SEQ ID NO: 5. In other embodiments, a probe or a nucleic acid primer is about 15, 16, 17, 18, 19, 20, 21, 22, 23, 24, 25, 26, 30, 35, 50 or 100 contiguous nucleotides represented within SEQ ID NO: 5.

In an embodiment, a probe consists essentially of a sequence of about 15-100 or 15-20 nucleotides capable of selectively binding to SEQ ID NO: 5.

In an embodiment, the experimental allergic encephalomyelitis (EAE) was used as an exemplary model system to evaluate the role of stat4 isoforms in regulating autoimmune disorders. EAE is a T cell-mediated autoimmune disease model of multiple sclerosis (MS). Signal transducer and activator of transcription 4 (Stat4) is a transcription factor activated by interleukin 12 (IL-12) and IL-23, two cytokines known to play important roles in the pathogenesis of EAE by inducing T cells to secrete IFN-α and IL-17 respectively. Therapeutic intervention or targeted disruption of Stat4 was effective in ameliorating EAE. A splice variant of Stat4 termed Stat4β has been characterized that lacks 44 amino acids at the C-terminus of the full length Stat4α. It was examined herein whether T cells expressing either isoform impacted the pathogenesis of EAE. Transgenic mice expressing Stat4 upon a Stat4-deficient background develop an exacerbated EAE compared to wild-type mice following immunization with MOGp35-55 peptide, while Stat4α transgenic mice have greatly attenuated disease. The differential development of EAE in transgenic mice correlates with increased IFNγ and IL-17 in Stat4β-expressing cells in situ, contrasting increased IL-10 production by Stat4α-expressing cells. It is shown herein that Stat4 isoforms differentially regulate inflammatory cytokines in association with distinct effects on the onset and severity of EAE. Stat4β transgenic mice developed an exacerbated EAE in association with an increased expression of inflammatory cytokines The Stat4α transgenic mice remain resistant to EAE, indicating that Stat4α is more efficient than Stat4α in mediating the pathogenesis of EAE.

### BRIEF DESCRIPTION OF THE DRAWINGS

FIG. 1 shows development of EAE in Stat4α and Stat4β transgenic mice. A, C57BL/6 wild type (WT), Stat4 deficient (Stat4−/−), Stat4α transgenic (Stat4α) and Stat4β transgenic (Stat4β) mice were induced to develop EAE by immunization with MOGp35-55 antigen. The clinical symptoms were scored every day in a blinded manner. The mean clinical score of all 10 mice from two different experiments is shown. The figure is representative of three independent experiments. B, Neural antigen-induced proliferation of spleen T cells from Stat4α and R transgenic mice in vitro. Spleen cells were isolated from C57BL/6 wild type (WT), Stat4 deficient (Stat4−/−), Stat4α transgenic (Stat44x), and Stat4β transgenic (Stat4β) mice on day 14 following induction of EAE. The cells were cultured with MOGp35-55 for 48 hrs and the proliferation measured by WST-1 assay.

FIG. 2 shows histology of CNS inflammation and demyelination in Stat4α and Stat4β transgenic mice with EAE. The spinal cord samples were isolated from C57BL/6 wild type (WT), Stat4 deficient (Stat4−/−), Stat4α transgenic (Stat4α) and Stat4β transgenic (Stat4β) mice on day 30 following induction of EAE. The transverse sections of cervical, upper thoracic, lower thoracic and lumbar regions of spinal cord were obtained and stained with LFB/PAS (luxol fast blue/periodic acid scriff) along with H&E (hematoxylin and eosin). The pathology of demyelination (left) and inflammation (right) in the spinal cord sections were visualized by microscopy and the representative 10× pictures are shown. The number of positive quadrants with inflammation and demyelination were scored and expressed as percentage over the total number of quadrants examined in the histogram.

FIG. 3 shows expression of effector T cell-derived inflammatory cytokines in the CNS, spleen and cultured spleen cells from Stat4α and Stat4β transgenic mice with EAE. Brain and spleen samples were isolated from C57BL/6 wild type (WT), Stat4α transgenic (Stat4α), and Stat4β transgenic (Stat4β) mice on day 14 following induction of EAE by immunization with MOGp3555 antigen. Total RNA was extracted from brain, spleen or spleen cells cultured with neural antigens and the expression of IFNγ, IL-17 and T-bet analyzed by qRT-PCR using GAPDH as internal control. The fold changes in the expression of cytokines in EAE mice were calculated based on naïve mice as control.

FIG. 4 shows expression of APC-derived inflammatory cytokines in the CNS and spleen of Stat4α and R transgenic mice with EAE. Brain and spleen were isolated from C57BL/6 wild type (WT), Stat4α transgenic (Stat4α) and Stat4β transgenic (Stat4β) mice on day 14 following induction of EAE by immunization with MOGp35-55 antigen. Total RNA was extracted from brain and spleen and the expression of IL-12p35, IL-12p40 and IL-23p19 analyzed by qRT-PCR using GAPDH as internal control. The fold changes in the expression of cytokines in EAE mice were calculated based on naïve mice as control.

FIG. 5 shows intracellular IFNγ and IL-17 in immune cells from Stat4α and R transgenic mice. Spleen, lymph node and brain cells were isolated from C57BL/6 wild type (WT), Stat4α transgenic (Stat4α) and Stat4β transgenic (Stat4β) mice on day 14 following induction of EAE. The cells were cultured with PMA+ionomycin for 6 hours before staining with IFNγ and IL-17 specific antibodies and analyzed by flow cytometry.

FIG. 6 shows neural antigen-induced secretion of IFNγ and IL-17 from Stat4α and R transgenic spleen cells in culture. Spleen cells were isolated from C57BL/6 wild type (WT), Stat4α transgenic (Stat4α) and Stat4β transgenic (Stat4β) mice on day 14 following induction of EAE. The cells were cultured with MOGp35-55 or Con A for 36 h, and the release of IFNγ (A) and IL17 (B) was analyzed by ELISA.

FIG. 7 shows differential regulation of IL-10 by Stat4 isoforms. A, Affymetrix Integrated Genome Browser analysis of Stat4 binding across the I110 locus using data from a Stat4 ChIPon-chip dataset. Bars indicate the intensity of Stat4-bound DNA hybridizing to oligonucleotides spanning −7.5 kb to +2.5 kb relative to the I110 transcriptional start. The exon-intron structure of I110 is indicated below the graph. B, Naïve, wild type and Stat4−/− CD4+ T cells were cultured under Th1 conditions for five days before re-stimulation with IL-12 (left) or anti-CD3 (right) for 24 hrs. IL-10 levels were examined in supernatants using ELISA. C, Naïve, wild type, Stat4α transgenic and Stat4β transgenic CD4+ T cells were cultured under Th1 conditions for five days before re-stimulation with anti-CD3 for 24 hours. IL-10 and IFNγ levels were examined in supernatants using ELISA. D, I110 mRNA levels were assessed in total RNA from spleens isolated from C57BL/6 wild type (WT), Stat4α transgenic (Stat4α) and Stat4β transgenic (Stat4β) mice on day 14 following induction of EAE by immunization with MOGp35-55 antigen by qRT-PCR using GAPDH as internal control. The fold changes in the expression levels were calculated based on naïve spleen. E, Spleen cells were isolated from C57BL/6 wild type (WT), Stat4α transgenic (Stat4α) and Stat4β transgenic (Stat4β) mice on day 14 following induction of EAE. The cells were cultured as in FIG. 6 and the production of IL-10 was analyzed using ELISA.

FIG. 8 shows T-cells expressing STAT4 isoforms have differential TNF-α production. A, CD4+CD62L+ T-cells from mice of the indicated genotypes were cultured under Th1 priming conditions (IL-12, anti-IL-4, a-CD3, a-CD28) with irradiated APCs (30 Gy) for five days. Every 24 hours, supernatants of the developing Th1 cells were collected from each genotype. Cell free supernatants were analyzed for IFN-γ production using ELISA. Results are represented as mean±SD. B, Cells cultured as in (A) for five days were stimulated for 24 hours and cell-free supernatants were analyzed for IFN-γ using ELISA. Results are represented as mean±SD and are representative of 3 independent experiments. C, CD4+CD62L+ T-cells were cultured as in (A) for five days. Cells were collected, washed, and stimulated with PMA and ionomycin in the presence of GolgiPlug before intracellular staining for the indicated cytokines. Data shown are gated on CD4+ cells. Numbers represent % of cells in the respective quadrant while numbers in parentheses represent the MFI of the x-axis. Results are representative of 3 independent experiments. D, RNA was isolated from Th1 cells cultured as in (A) following 4 hours of treatment with anti-CD3. Quantitative PCR was performed for Tnfa mRNA and normalized for P2m expression. Results are relative to WT cells. E, Cells cultured under Th1 priming conditions for five days were stimulated in the indicated condition for 24 hours before cell-free supernatants were collected for analysis of TNF-α. F, Cells cultured as in (A) for five days were stimulated for 24 hours and cell-free supernatants were analyzed by ELISA for TNF-a and IL-2. Results are represented as mean±SD and are representative of 2-4 independent experiments. *, significantly different (p<0.05) from wild-type, Stat4α, and Stat4−/− Th1 cultured cells using unpaired Student's T-test.

FIG. 9 shows activation kinetics of the STAT4 isoforms during Th1 differentiation. A, Naïve CD4+ T cells freshly isolated (0 time point) or cultured in Th1 conditions for 24, 48, or 72 hours were collected for intracellular staining with anti-pStat4. Results are representative of 2 independent experiments. Numbers in quadrants represent % of pSTAT4+ T cells. B, Total cell extracts from WT and STAT4 transgenic T cells were immunoblotted for STAT4 protein levels at day 1, 2 and 5 of Th1 differentiation. Data is presented as arbitrary units of densitometry normalized to actin expression and relative to WT day 1 in the left panel or WT day 5 in the right panel. C, Th1 cells cultured for 5 days were washed and stimulated with IL-12 for the indicated time points before being intracellular stained for pSTAT4. Data are shown for the averages of duplicate samples of representative data. D, Th1 cells were stimulated with IL-12 and IL-18 for 24 hours and cell-free supernatants were analyzed for IFN-γ using ELISA. Results are shown as mean±SD.

FIG. 10 shows effects of STAT4α and STAT4β expression on Th17 differentiation. A, CD4+CD62L+ T-cells were cultured in the presence of TGF-β1, IL-6, IL-23, anti-IL-4, and anti-IFN-γ for 5 days. Cells were collected, washed, and stimulated with plate-bound a-CD3 or PMA and Ionomycin (P+I) in the presence of Golgi-Plug before intracellular staining for the indicated cytokines. CD4+ cells were gated and the results were plotted as indicated. Numbers represent % of cells in the respective quadrant. Results are representative of 2 independent experiments. B, Total CD4 T cells were cultured for five days in the presence of IL-23 before restimulation with anti-CD3 and assessing production of IL-17A using ELISA. Results are shown as mean±SD and are representative of 3 independent experiments. C, T cells cultured as in (B) were stimulated with IL-23 and IL-18 for 24 hours and cell-free supernatants were analyzed by ELISA for IL-17A. Results are shown as mean±SD of results from 2-4 independent experiments.

FIG. 11 shows that STAT4α and STAT4β mediate inflammatory bowel disease. A, The change of weight over time is expressed as percent of the original weight. Data represent the mean±SEM of each group (7-10 mice per group). Mice were sacrificed 14 weeks after T-cell reconstitution. *, CD45RBlow cells are significantly different (p<0.05) from CD45RBhigh WT, STAT4α or STAT4β using 2-way ANOVA and unpaired Student's Ttest post-hoc. B, MLN single-cell suspensions were counted and surface stained for CD4 and analyzed by FACS. Absolute cell numbers were calculated from % of CD4+ cells and cell counts (left panel). QPCR was performed for STAT4 using cDNA made from MLN RNA (right panel). C, Gross appearance of representative colon from each group as indicated. D, Representative photomicrographs (100×) of colon from mice of the indicated group were stained with H&E. E, The mean histological scores±SEM for the SCID mice reconstituted with the CD4+ T-cells as indicated with STAT4α or STAT4β signifying histological scores from the SCID mice reconstituted with the CD45RBhigh subset and the low signifying histological scores from the SCID mice reconstituted with the CD45RBlow subset. *, p<0.05 where STAT4β is significantly different from both STAT4α and the CD45RBlow subset using the Mann-Whitney U-test.

FIG. 12 shows cytokine production from STAT4α- and STAT4β-expressing T cells ex vivo. (A and B) Cells were isolated and stimulated as described in Materials and Methods and concentration of cytokines were determined by ELISA and are displayed as mean±SEM (Stat4α n=9; Stat4β n=10). *, p<0.05, ** p<0.02 using Unpaired Student's T-test. C, Cells were isolated and stimulated as described in Materials and Methods. The concentration of cytokines were determined by ELISA and are displayed as mean±SD of pooled MLNs from the SCID mice reconstituted with the CD45RBhigh subset of the indicated STAT4 isoform. *, p<0.05 using unpaired Student's T-test.

FIG. 13 shows increased lamina propria neutrophil infiltration correlates with increased GM-CSF levels seen in the SCID mice reconstituted with the STAT4β isoform. A, PMN scores were determined as described in Materials and Methods. Data are presented as mean±SEM. *, p<0.05 using Mann-Whitney U test. B, Single cell suspensions from MLNs were pooled from the indicated mice, stimulated with anti-CD3 for 72 hours and cell-free supernatants were analyzed using ELISA for GM-CSF. Data are presented as mean±SD. *, p<0.05 using unpaired Student's T-test.

FIG. 14 shows that STAT4β Th1 cells are programmed to secrete more GM-CSF than STAT4α Th1 cells. A, CD4+CD62L+ T-cells were primed for Th1 differentiation using the same conditions as in FIG. 1. After five days, cells were stimulated for 24 hours and cell-free supernatants were analyzed by ELISA for GM-CSF. Results are represented as mean±SD and are representative of 3 independent experiments. *, p<0.05 using unpaired Student's T-test. B, Cells cultured under Th17 conditions as in FIG. 3A for five days were stimulated for 24 hours and analyzed by ELISA for GM-CSF production. Results are presented as mean±SD and are representative of 2 independent experiments.

FIG. 15 shows nucleic acid and amino acid sequences for Stat4α and Stat4β isoforms and the different exons.

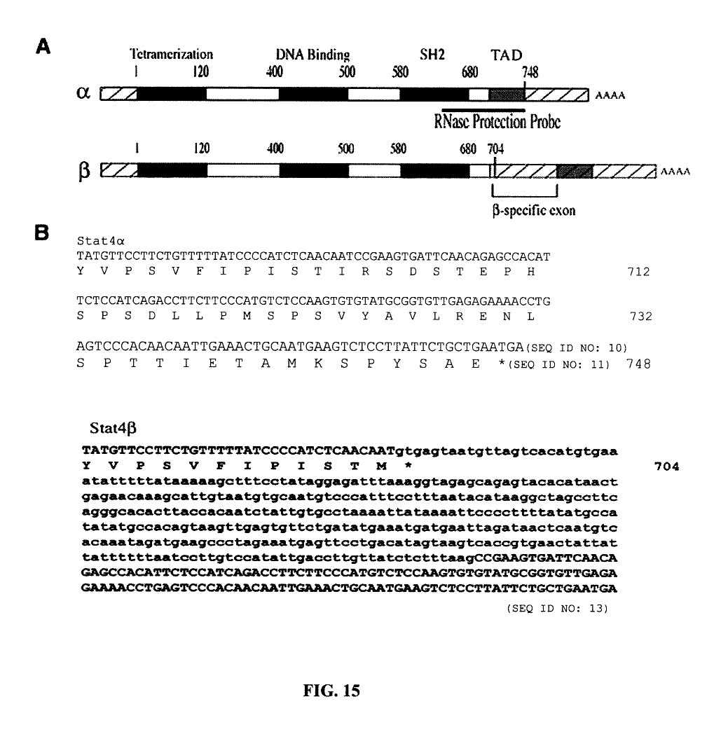

FIG. 16 shows the STAT4β/STAT4α ratio in two different control groups (the left infants; the right a group that is age-matched to the patients), CD and UC patient groups. The ratio is consistently elevated in the patient samples. The right graph shows the Crohn's disease severity score with the beta/alpha ration divided into groups higher or lower than 10. The data indicates that patients with higher ratios have more severe disease symptoms.

### DETAILED DESCRIPTION

In the present disclosure the ability of Stat4α and Stat4β transgenic T cells to mediate inflammatory disease was tested. In a model of colitis, the Stat4β isoform appeared to be more potent in generating inflammation than the Stat4α isoform. This correlates with an increased production of inflammatory cytokines, particularly TNFα. Similarly, in an Experimental Autoimmune Encephalomyelitis (EAE) model of central demyelination, the Stat4β transgenic mice get worse disease than wild type mice while the Stat4α transgenic mice get less severe disease than wild type mice. Stat4-deficient mice are relatively protected from disease in each model.

Knowledge of Stat4 alpha/beta ratios may provide clinicians with information to decide a patient's risk for developing inflammatory diseases, predicting the severity (and for taking precautionary steps to avoid undesirable outcomes), and/or determining potential response/non-response to immunosuppressive treatments such as anti-TNF therapy for diseases including rheumatoid arthritis, lupus, inflammatory bowel disease, multiple sclerosis and the like. Because immunosuppression therapy often increases an individual's risk to sepsis, tuberculosis and other infectious diseases, predictive evidence for efficacy of an immunosuppressive treatment such as anti-TNF therapy helps reduce the overall incidence of life-threatening infections.

Stat4α and Stat4β transgenic mice were used to define the ability of Stat4 isoforms to mediate CNS inflammation and demyelination in the EAE model of MS. Stat4β transgenic mice develop exacerbated EAE compared to wild type mice, while the Stat4α transgenic mice developed mild EAE. The exacerbation of EAE in Stat4β transgenic mice associated with lower levels of IL-10 production and increased expression of inflammatory cytokines including IFNγ and IL-17 compared to Stat4α transgenic mice. These findings highlight the fact that Stat4 isoforms play distinct roles in the pathogenesis of EAE.

Each isoform of Stat4 is sufficient to program Th1 development through both common and distinct subsets of target genes. The ability of these isoforms to mediate inflammation in vivo has not been examined. Using a model of colitis that develops following transfer of CD4⁺ CD45RBhi T-cells expressing either the STAT4α or STAT4β isoform into SCID mice, it was determined that while both isoforms mediate inflammation and weight loss, STAT4β promotes greater colonic inflammation and tissue destruction. This correlates with STAT4 isoform-dependent expression of TNF-α and GM-CSF in vitro and in vivo, but not Th1 expression of IFN-γ or Th17 expression of IL17, which were similar in STAT4α- and STAT4β-expressing T cells. Thus, higher expression of a subset of inflammatory cytokines from STAT4β-expressing T cells correlates with the ability of STAT4β-expressing T cells to mediate more severe inflammatory disease.

To test the ability of Stat4 isoforms to mediate inflammatory disease, a model wherein CD4+CD45RBhi T cells expressing either Stat4α or Stat4β were transferred into SCID recipients to induce colitis was used. Stat4β mediated more severe inflammation and this correlated with the ability of Stat4β-expressing T cells to secrete higher levels of a subset of Th1 inflammatory cytokines in vitro and in vivo. Thus, Stat4β, an isoform that lacks the C-terminal transactivation domain, is more efficient than Stat4α in promoting inflammation in vivo.

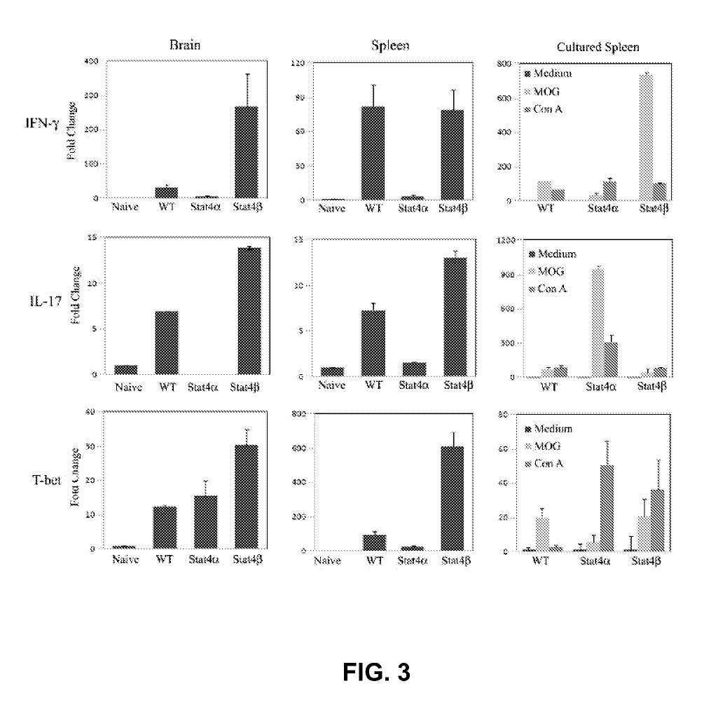

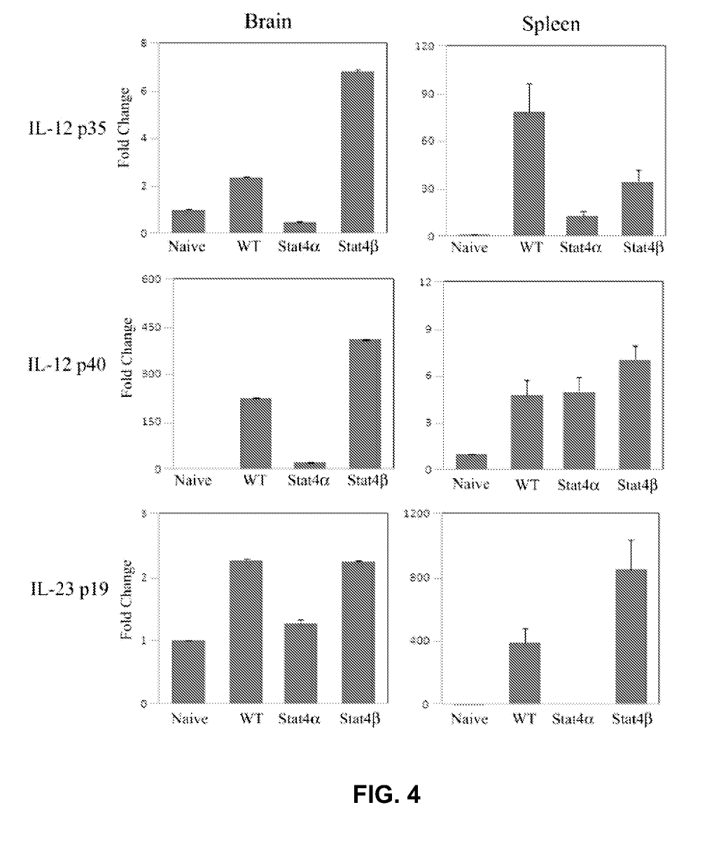

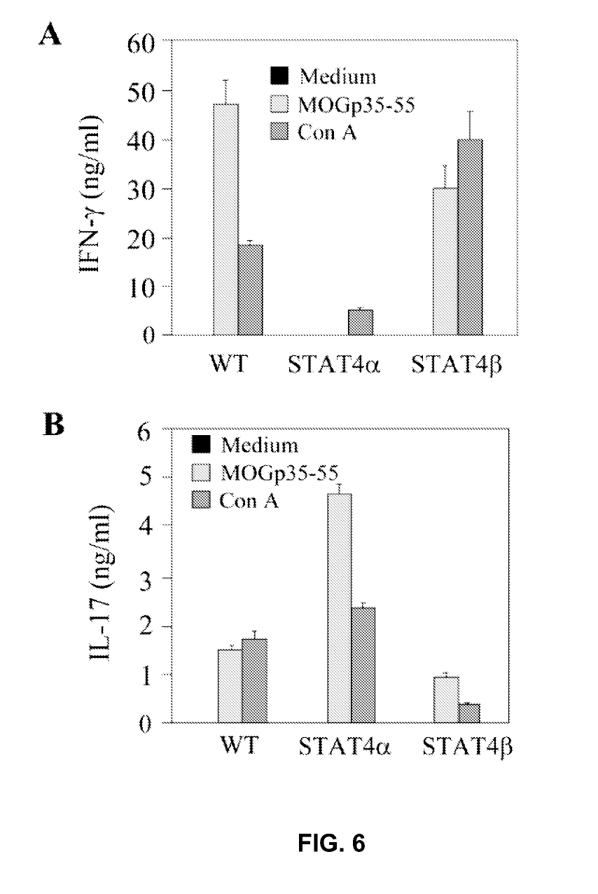

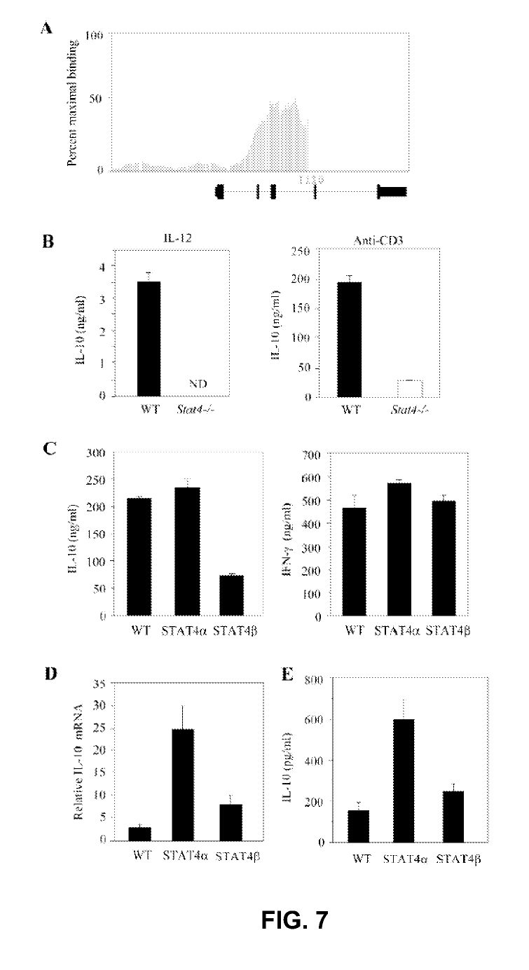

Data provided herein demonstrate that T cells expressing Stat4β are much more efficient in mediating inflammation than T cells expressing the Stat4α isoform. Stat4β transgenic mice develop much more severe disease with greater levels of demyelination than those observed in Stat4α transgenic mice or wild type mice. The mechanism for this increased disease may include the altered cytokine environments observed in the transgenic mice. While Stat4α and Stat4β transgenic cells are equally capable of becoming IFNγ- or IL-17-secreting cells in vitro, Stat4α transgenic cells have increased levels of IL-10 production (FIG. 7). Similar increased IL-10 production was observed in Stat4α transgenic mice with EAE in vivo and ex vivo (FIG. 7). The lower levels of IL-10 produced in Stat4β transgenic mice are associated with increased IL-12 and IL-23 mRNA levels in CNS and spleen tissue, and increased IFNγ expression in tissue and from antigen-stimulated Stat4β-expressing cells compared to those observed in Stat4α-expressing cells (FIGS. 3-7). The lower levels of IFNγ produced by Stat4α transgenic cells compared to Stat4β transgenic cells may, at least in part, be responsible for the observed increases in IL-17 from Stat4α transgenic cells in the periphery (FIGS. 4 and 6). Data from a ChIP-on-chip assay are provided herein that Stat4 directly binds IL-10, and show that acute stimulation of Th1 cells with IL-12 results in IL-10 production from wild type but not Stat4−/− cells (FIG. 7). Moreover, Stat4α, but not Stat4β, can mediate the programming of the IL-10 gene for increased expression in Th1 cultures. Thus, while Stat4α can rescue Stat4-deficiency in vitro and compensates for some in vivo Stat4 functions, altered cytokine profiles from these cells limit their ability to promote the development of EAE. As IL-10 is critical regulator of inflammation in EAE, the increased IL-10 production in Stat4α transgenic mice (FIG. 7) provides a mechanism how Stat4 isoforms differentially regulate the pathogenesis of EAE. These results also indicate that modulating the splicing between the alpha and beta isoforms of Stat4 has therapeutic value for inflammatory diseases.

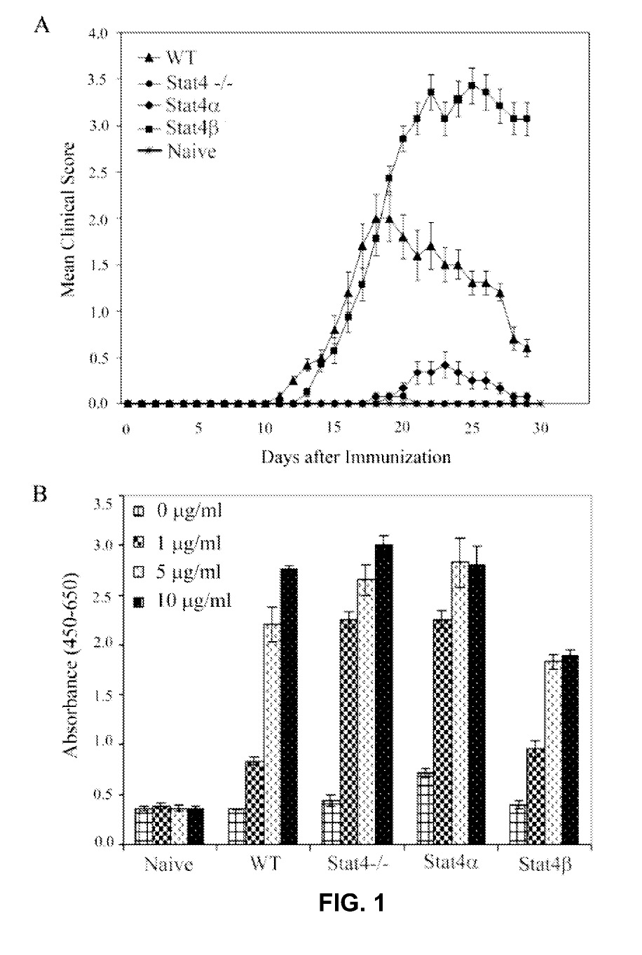

It is also possible that other Stat4α- or Stat4β-specific functions might be important for the pathogenesis of EAE. While both isoforms could mediate Th1 development in vitro, Stat4β-expressing cells produced slightly less IFNγ in response to IL-12. Since there was more severe disease in Stat4β transgenic mice, it seems unlikely this contributes to the level of disease. Stat4β transgenic cells had much higher proliferation than Stat4α or wild type cells in a pattern that paralleled the severity of disease. However, MOGp35-55-specific proliferation indicates that there is no significant increase in the overall number of antigen-reactive T cells in EAE (FIG. 1). Similarly, intracellular cytokine staining did not show dramatic differences among the percentages of cytokine-positive cells, though IFNγ was increased in the Stat4β transgenic cells. As noted above, this is more likely to result from changes in the balance of IL-10 and IFNγ production, and their resulting effects on IL-17 production. However, there may be additional genes that are differentially regulated by Stat4 isoforms which may also contribute to the development of inflammatory diseases.

Data provided herein further demonstrates that Stat4 expression in T cells may be sufficient to mediate inflammatory immunity. The Stat4α and Stat4β transgenes are expressed from a CD2 locus control region that promotes transcription primarily in T cells, with considerably lower expression in other lymphoid cells. The transgenic mice have been backcrossed to the Stat4−/− background so that the Stat4 isoforms are expressed in T cells but not other cells in the mouse. As the transgenic mice in this study lack Stat4 in any myeloid compartment, results indicate that Stat4 expression in non-lymphoid cells may not be required for the development of EAE. It may be possible to alter Stat4 function by modulating the splicing of Stat4 isoforms and thus altering the ability of immune cells to mediate disease.

TNFα and GM-CSF production are Stat4-dependent in Th1 cells and that Stat4β more effectively programs the secretion of these cytokines following subsequent antigen receptor stimulation. Thus, Stat4 isoforms may have differing roles in the development of inflammation.

As Stat4 has been implicated as a pathogenic factor in Th1 and Th17-mediated autoinflammatory diseases, including IBD, an IBD model system where colitis is induced in SCID mice upon reconstitution with CD4+CD45RBhigh T cells was chosen to test the roles of Stat4 isoforms in disease. This model system has the advantage of being able to directly test the ability of T-cells expressing the Stat4 isoforms to mediate pathogenesis with minimal manipulation after reconstitution.

Although Crohn's disease and ulcerative colitis (UC) are the most common forms of inflammatory bowel disease (IBD), other forms include for example, collagenous colitis, lymphocytic colitis, ischaemic colitis, diversion colitis, Behçet's syndrome, infective colitis, and indeterminate colitis. In young adults that include pediatric patients of ages under 17, one or more forms of the above-listed IBD may be more common.

The ratio of Stat4β/stat4α expression levels may vary depending upon the disease or tissue or the inflammatory response being investigated. For example, the ratio (β/α) may range from about 0.1 to about 10 or about 0.2, 0.3, 0.4, 0.5, 1.5, 2.0, 3.0, 4.0, 5.0, 6.0, 7.0, 8.0, 9.0, 10.0, 15.0, 20.0, 25.0, 30.0, 40.0, 50.0 or higher. Depending on the conditions, the ratio may also range less than about 5.0 or 2.0 or 3.0. It may also range about 0.1-2.00. Depending on the sensitivity of the detection system and the relative units, the ratio may vary over a broader range. In other words, the scope of the disclosure is not limited to the explicit ratios disclosed herein. A skilled artisan, based on the disclosure and guidance herein may readily evaluate the ratio by determining the relative expression levels of α and β isoforms of Stat4 in a cell, plurality of cells, tissue, for a disease of interest. In some instances, one of the isoforms e.g., α or β may not be expressed to a detectable level or expressed to an extremely low level. Accordingly, appropriate ratios or the mere absence of a particular isoform may be used as predictive or diagnostic markers for an inflammatory response of interest. In an embodiment, the relative expression status of the α or the β isoform of Stat4 is a marker useful for evaluating, diagnosing or predicting the efficacy of anti-inflammatory therapies.

The term correlating in reference to a parameter, e.g., expression levels of Stat4α/β in a sample generally includes any method of relating levels of expression of markers to a standard or a reference value for the assessment of the diagnosis, prediction of an immune disorder including Crohn's disease, ulcerative colitis, IBD and/or assessment of efficacy of clinical treatment, e.g., anti-TNF therapy, identification of a patient group that responds to a particular therapy, selection of a subject for a particular treatment, and monitoring of the progress of treatment with an anti-TNF therapy.

In an embodiment, the Stat4β:Stat4α expression ratio are correlated with a well-known scoring metric, e.g., Pediatric Crohn's Disease Activity Index (PCDAI), Hyams et al. (1991), Development and validation of a pediatric Crohn's disease activity index. J Pediatr Gastroenterol Nutr; 12:439-447, incorporated herein by reference. See also, Harvey R, Bradshaw J (1980). A simple index of Crohn's-disease activity. Lancet 1 (8167): 514; Yoshida et al., (1999), The Crohn's Disease Activity Index, its derivatives and the Inflammatory Bowel Disease Questionnaire: a review of instruments to assess Crohn's disease. Can J. Gastroenterol.; 13(1):65-73; and Turner et al., (2007) Development, Validation, and Evaluation of a Pediatric Ulcerative Colitis Activity Index: A Prospective Multicenter Study, Gastroenterology; 133(2): 423-432, all incorporated by reference herein.

Anti-tumor necrosis factor (TNF) strategies for immune disorders, include for example chimeric monoclonal (infliximab), humanized monoclonal (CDP571 and the PEGylated CDP870) and fully human monoclonal (adalimumab) antibodies against TNF, p75 fusion protein (etanercept), p55 soluble receptor (onercept) and small molecules such as MAPkinase inhibitors. Infliximab is often prescribed in treating active Crohn's disease patients that do not respond to or intolerant of conventional therapies (e.g., steroidal or other non-steroidal treatments). In patients who fail to respond therapy with corticosteroids and immunosuppressive therapy and are poor candidates for surgery, and patients with fistulizing disease, where infliximab therapy is chosen, regular maintenance therapy with infliximab may be required. Afelimomab (also known as Fab2 or MAK 195F) is an anti-TNF-α monoclonal antibody. Certolizumab pegol is a monoclonal antibody directed against tumor necrosis factor alpha. It is a PEGylated Fab′ fragment of a humanized TNF inhibitor monoclonal antibody.

Nucleic acid or nucleic acid sequence or polynucleotide or polynucleotide sequence refers to the sequence of a single- or double-stranded DNA or RNA molecule of genomic or synthetic origin, i.e., a polymer of deoxyribonucleotide or ribonucleotide bases, respectively.

Detection of expression of Stat4 isoforms or fragments thereof and any other genes include detecting the expression of RNA, its reverse transcribed cDNA and the resulting protein products thereof. For example, Stat4 isoform RNA can be detected using in situ RT-PCR or in vitro RT-PCR or standard PCR or through any hybridization techniques that involve Stat4α and β isoform specific probes or primers. RNA expression can also be quantified by any known quantification PCR (qPCR) and competitive PCR technology (see e.g., Nolan T, Hands R E, Bustin S A (2006). “Quantification of mRNA using real-time RT-PCR.”. Nat. Protoc. 1: 1559-1582). Microarrays are also useful in quantifying gene expression. RNA can be extracted from a suitable tissue or a cell population, converted into cDNA and quantified using any known method. RNA can also be directly quantified in situ, within the tissue or the cell population itself. Probes specific to Stat4 isoforms may display high stringency hybridization. Similar techniques are also useful in determining the expression pattern and quantity of any other genes.

High stringency hybridization conditions or highly stringent hybridization include at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. These conditions are used for detecting expression levels of Stat4α/β isoforms using the probes disclosed herein or to identify additional specific probes for expression detection. Moderately stringent conditions may be obtained by varying the temperature at which the hybridization reaction occurs and/or the wash conditions as set forth above.

Nucleic acid probes are used to detect and/or quantify the presence of Stat4 transcript in a sample, e.g., as hybridization probes, or to amplify Stat4 transcript or partial regions thereof in a sample, e.g., as a primer. Probes have a complimentary nucleic acid sequence that selectively hybridizes to the desired target nucleic acid sequence. The hybridization probe must have sufficient identity with the target sequence, i.e., at least 70%, e.g., 80%, 90%, 95%, 98%, or 99% or more identity to the target sequence. The probe sequence is also sufficiently long so that the probe exhibits selectivity for the target sequence over non-target sequences. For example, the probe will be at least about 20, e.g., 25, 30, 35, 50, 100, 200, 300, 400, 500, 600, 700, 800, 900 or more, nucleotides in length. Probes include primers that refer to a single-stranded oligonucleotide probe that can act as a point of initiation of template-directed DNA synthesis using methods such as PCR (polymerase chain reaction), LCR (ligase chain reaction), etc., for amplification of a target sequence.

Nucleic acid probes and oligonucleotide primers may also contain modified nucleotides to enhance stability and/or specificity.

Stat4 gene expression may be measured using any suitable method such as for example, western/immunoblot methods that detect the protein product. Western/immunoblot uses gel electrophoresis to separate native or denatured Stat4 proteins by the length of the polypeptide (i.e., α and the β isoforms) or by the 3-D structure of the protein (native/non-denaturing conditions). The proteins are transferred to a membrane (e.g., nitrocellulose), where they are detected using antibodies specific to the target protein or polypeptides

Stat4 gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence Stat4 α and β polypeptides or against a synthetic peptide of Stat4α or β based on the DNA sequences provided herein.

Stat4 isoforms (proteins) or fragments or polypeptides thereof detection can also be carried in situ or after extraction. Antibodies including monoclonal antibodies against a specific Stat4 isoform are useful in quantifying Stat4α and Stat4β protein levels. Standard immuno histochemistry techniques are capable of detecting the presence and the amount of Stat4α/β antibodies either directed to the full length Stat4 isoforms or a peptide fragment thereof. The antibodies include monoclonal or polyclonal antibodies. Anti-Stat4 antibodies are available. See e.g., Hoey et al., (2003), incorporated by reference in its entirety, including Stat4α and β specific sequences and antibodies that recognize both the isoforms.

The term prognosis generally refers to a forecast or prediction of the probable course or outcome of an immune disorder.

The term predictive marker generally refers to a factor that indicates sensitivity or resistance to a specific treatment. Thus, a predictive marker provides a measure of likelihood of response or resistance to a particular therapy.

For example, ratio of Stat4α/Stat4β expression is used as both a prognostic marker (e.g., better response for anti-TNF therapy) and as a predictive marker (greater likelihood of response to anti-TNF therapy) for one or more of the immune disorders.

A sample (also used as a biological sample or tissue or cell sample) generally includes a variety of sample types obtained from an individual and can be used in a diagnostic or monitoring assay. The term includes blood and other liquid samples of biological origin, solid tissue samples such as a biopsy specimen or tissue cultures or cells derived therefrom, and the progeny thereof. A suitable tissue sample is for example, a sample of tissue from the lining of the intestine or biopsy of the gastric antrum. The term also includes samples that have been manipulated in any way after their procurement, such as by treatment with reagents, solubilization, or enrichment for certain components, such as proteins or polynucleotides, or embedding in a semi-solid or solid matrix for sectioning purposes. The term biological sample encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; amniotic fluid, peritoneal fluid, or interstitial fluid. The biological sample may contain compounds which are not naturally intermixed with the tissue in nature such as preservatives, anticoagulants, buffers, fixatives, nutrients, antibiotics, or the like. In an embodiment, a suitable sample includes T-cells.

The term gene generally refers to any polynucleotide sequence or portion thereof with a functional role in encoding or transcribing a protein or regulating other gene expression. The gene may include all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

The term antibody generally refers to intact monoclonal antibodies, polyclonal antibodies, multispecific antibodies (e.g. bispecific antibodies) formed from at least two intact antibodies, and antibody fragments that are capable of recognizing Stat4 expression products.

The term treatment or therapy generally relates to an approach for obtaining beneficial or desired clinical results. This includes: inhibiting and/or relieving to some extent one or more of the symptoms associated with the disorder, decreasing symptoms resulting from the disease, increasing the quality of life of those suffering from the disease, decreasing the dose of other medications required to treat the disease, delaying the progression of the disease, and/or mitigating the side-effects associated with the therapies.

Some of the symptoms for Crohn's disease include for example, abdominal pain, often in the right lower quadrant, and diarrhea. Rectal bleeding, weight loss, arthritis, skin disorders, eye inflammation and fever may also occur. Bleeding may be serious and persistent, leading to anemia. Children with Crohn's disease may have delayed development and stunted growth.

Symptoms for ulcerative colitis include for example, include rectal bleeding and diarrhea. Symptoms may vary depending on the amount of the colon and rectum that are inflamed and the intensity of inflammation. Ulcerative colitis are generally classified according to the location and the extent of inflammation, including for example, ulcerative proctitis (inflammation that is limited to the rectum), proctosigmoiditis (inflammation of the rectum and the sigmoid colon), pancolitis or universal colitis (inflammation affecting the entire colon), and fulminant colitis (severe form of pancolitis).

The identification and analysis of molecular markers (expression levels of Stat4α and β), have numerous therapeutic and diagnostic purposes. Clinical applications include, for example, selection of therapy including dosage, prediction of a therapeutic response; prediction of efficacy of therapy; monitoring of patients' response trajectories (e.g., prior to onset of disease) and/or after the onset of disease; prediction of adverse response; monitoring of therapy associated efficacy and toxicity; prediction of probability of occurrence; recommendation for prophylactic measures; and detection of recurrence.

The molecular markers disclosed herein may be detected using any suitable conventional analytical technique including but not limited to, immunoassays, protein chips, multiplexed immunoassays, complex detection with aptamers, chromatographic separation with spectrophotometric detection, mass spectroscopy, cDNA microarrays, and nucleic acid probe hybridization.

Stat4α/Stat4β expression in the test biological sample (i.e., the biological sample from a patient having an immune disorder or suspected of having the immune disorder) may be compared to a suitable control sample, as is well known in the art. Exemplary controls include comparable normal samples (e.g., normal tissue or cells of the same type as present in the test biological sample), matched normal samples from a similar patient, universal control samples, or a normal reference value (also termed a control reference value). The term control or control sample may also encompass a normal reference value. Methods for comparison of expression levels (such as presence or absence of or amount of expression) are known in the art.

As discussed herein, expression in a biological sample can be detected by a number of methods which are well-known in the art, including but not limited to, immunohistochemical and/or Western analysis, biochemical enzymatic activity assays, in situ hybridization, Northern analysis and/or PCR analysis of mRNAs, and genomic Southern analysis (to examine, for example, gene deletion or amplification), as well as any one of the wide variety of assays that can be performed by gene, protein, and/or tissue array analysis.

Consists essentially or consisting essentially of refers to a portion of Stat4α or Stat4β nucleic acid or amino acid sequences that are specific to each of the isoforms and may contain other non-specific sequences that do not alter the specific detection capabilities.

### EXAMPLES

The following examples are to be considered as exemplary and not restrictive or limiting in character and that all changes and modifications that come within the spirit of the disclosure are desired to be protected.

### Example 1

### Determining Expression Levels of Stat4α and Stat4β

Samples from patients exhibiting inflammatory bowel disease-like symptoms were analyzed for the expression levels of Stat4 isoforms. The primer sequences used to amplify and quantify the Stat4 isoforms are as follows:

Stat4ex17f
(SEQ ID NO: 6)
(5′- TAT CCT GAC ATT CCC AAA GAC -3′)
(Common forward primer)
Stat4ex19r
(SEQ ID NO: 7)
(5′- CTC TCA ACA CCG CAT ACA CAC -3′)
(α-specific reverse primer)
Stat4βr
(SEQ ID NO: 8)
(5′ GAC TTA CTA TGT CAG GAA CTC -3′)
(β-specific reverse primer).

This example demonstrates that tested samples from patients suffering from Crohns' disease of ulcerative colitis exhibit different Stat4β/Stat4α ratio compared to the control group. Stat4β/Stat4α ratios are useful in predicting efficacy of anti-TNF therapy for immune disorders.

### Example 2

### Clinical Investigation of Stat4 Beta/Alpha Ratios in Patients

This example illustrates protocols and experimental design to collect data for testing that patients with active Crohn's Disease, and/or ulcerative colitis have a higher β/α ratio than children who are healthy and there will be a change in the β/α ratio in response to corticosteroids or Infliximab treatment.

For example, in an embodiment, peripheral blood samples are purified over Ficoll to isolate mononuclear cells. Cells are initially frozen in liquid nitrogen aliquoted among 2-4 vials depending on cell number isolated. Once a sufficient number of samples has been collected, 5-10 samples are thawed and RNA is isolated using Trizol. RNA is then used as a template to make cDNA. The cDNA is used in a PCR reaction using primers specific for either the alpha or beta isoform of Stat4 (see Example 1 for an exemplary set of primers). PCR may be performed either semi-quantitatively, using dilutions of the cDNA to quantify mRNA levels, or using real time PCR for quantitative analysis. Relative levels are recorded and when sufficient numbers of samples have been examined, data are used for statistical analysis.

Peripheral blood samples may also be obtained for IL-12, IL-17 and TNFα levels, Sed rate (ESR) and Hematocrit. If the patients involve children, for example, the pediatric Crohn's disease activity index, the Rachmilewitz Clinical Activity index and the Colitis Symptom Score may also be assessed. See also D'Haens et al. (2007), A review of activity indices and efficacy end points for clinical trials of medical therapy in adults with ulcerative colitis. Gastroenterology; 132(4763-786. The above mentioned scores and indices may also be assessed at suitable intervals, e.g., 2-weeks. A signed consent/assent statement and completed eligibility checklist to include the subjects age, gender, and listing of prescription and over the counter medications are obtained from the subject/subject's family prior to analysis.

Descriptive statistics are calculated including medians, means and standard deviations of the measurements as appropriate for each of the three groups. No parametric distribution may be assumed for β/α ratio, the primary outcome of interest. A two sample Wilcoxon Rank Sum test may be used to assess if patients with active Crohn's Disease, and/or ulcerative colitis have a higher β/α ratio than patients who are healthy. To evaluate the change in the β/α ratio at two weeks post treatment from baseline in response to for example, corticosteroids or Infliximab treatment, Wilcoxon Signed Rank test may be performed. Graphical presentation may also be used to show the patient specific change in β/α ratio. The point estimate and 95% confidence interval for the mean of the β/α ratio may be reported on the log scale by group.

### Example 3

### Analysis of Stat4 Beta/Alpha Isoform Ratio in IBD

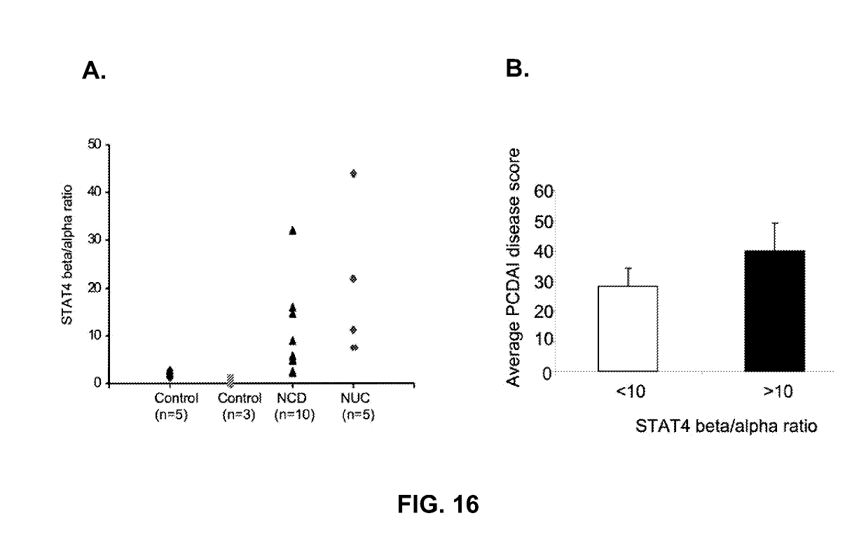

The expression of Stat4α and β isoforms in patients suffering from IBD was investigated. As shown in FIG. 16, the graph (FIG. 16A) shows the STAT4β/STAT4α ratio in two different control groups (the left infants; the right a group that is age-matched to the patients), Crohn's disease (CD) and ulcerative colitis (UC) patient groups. The STAT4β/STAT4α ratio is consistently elevated in the patient samples. The graph (FIG. 16B) shows the Crohn's disease severity score with the β/α ration divided into groups higher or lower than 10. This example indicates that higher STAT4β/STAT4α ratios correlate to the disease symptoms, e.g., Crohn's disease.

### Example 4

### Stat4β Transgenic Mice Develop an Exacerbated EAE

To study the distinct roles played by Stat4 isoforms in autoimmune disease, the development of EAE in transgenic mice was examined that express Stat4α and Stat4β directed by the CD2 locus control region backcrossed to a Stat4-deficient background and compared with wild type and Stat4−/− mice. As shown in FIG. 1A, Stat4β transgenic mice developed an exacerbated EAE compared to wild type mice. The day of onset and MCS in Stat4β transgenic mice was similar to wild type mice in the early phase of EAE, but the MCS continued to worsen in Stat4β transgenic mice in the later phase of EAE. In contrast, Stat4 deficient mice remained resistant to EAE and Stat4α transgenic mice developed mild EAE with delayed onset and earlier remission than the Stat4β transgenic and wild type mice (FIG. 1A). The Stat4β transgenic mice also showed a significant increase in AUC, MMCS and AMCS than the wild type and Stat4α transgenic mice. These results show that Stat4β transgenic mice develop an exacerbated EAE compared to Stat4α transgenic or wild type mice and suggest the distinct abilities of Stat4 isoforms to mediate the pathogenesis of EAE.

To confirm that Stat4α transgenic and Stat4−/− mice were sensitized to MOGp35-55 peptide, the antigen-induced T cell proliferation ex vivo was measure. As shown in FIG. 1B, in vitro culture of spleen cells from wild type, Stat4-deficient, Stat4α transgenic and Stat4β transgenic mice showed a dose-dependent proliferation in response to MOGp35-55 antigen with the Stat4β transgenic mice displaying a slightly decreased proliferation compared to cells from mice of the other genotypes. These results indicate that the lack of disease in Stat4α transgenic and Stat4−/− mice is not due to the lack of development of MOG-specific T cell responses.

### Example 5

### Stat4β Transgenic Mice Develop Severe Inflammation and Demyelination in the CNS

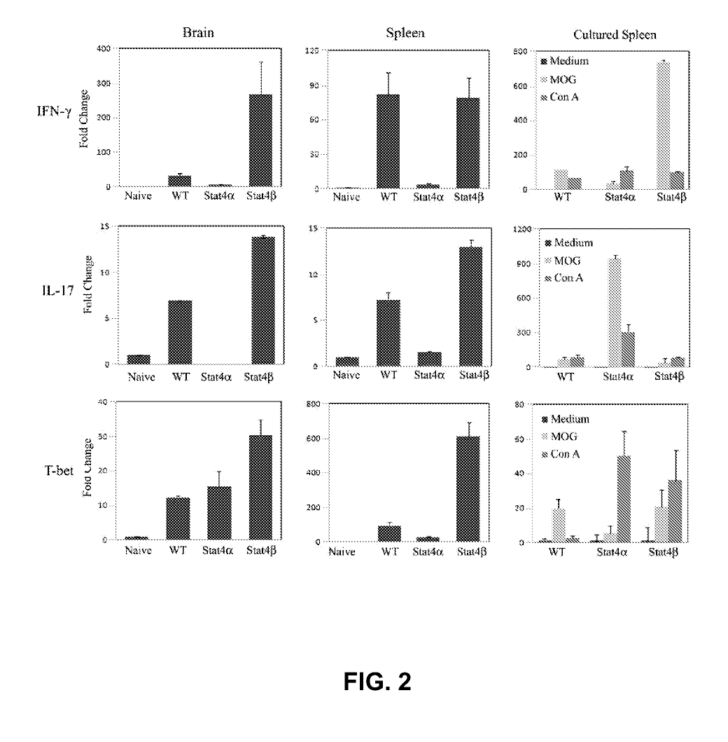

To further establish the differential regulation of EAE by Stat4 isoforms, the pathology of CNS inflammation and demyelination were examined. As shown in FIG. 2, the wild type mice with EAE showed extensive myelin loss (demyelination) associated with infiltration of immune cells (inflammation) in the spinal cord. When compared with wild type, the Stat4β transgenic mice with EAE showed a significant increase in the extent of inflammation and demyelination in the spinal cord. However, the Stat4−/− and Stat4α transgenic mice induced to develop EAE showed no sign of inflammation or demyelination in the CNS. Therefore, T cells expressing Stat4β caused more CNS pathology compared to T cells lacking Stat4 or those expressing Stat4α.

Histological analyses revealed that wild type mice with EAE display 18% and 21% spinal cord quadrants positive for demyelination and inflammation, respectively (FIG. 2). When compared with wild type mice, the Stat4β transgenic mice developed severe pathology with 71% (4 fold increase; p<0.01) and 61% (3 fold increase; p<0.01) spinal cord quadrants positive for demyelination and inflammation, respectively. The Stat4α transgenic mice developed very mild CNS pathology with 3% and 1.47% spinal cord quadrants positive for demyelination and inflammation respectively. The Stat4−/− mice failed to show any symptoms of CNS pathology (FIG. 2). These results indicate that the clinical symptoms of EAE correlate with the pathology of CNS inflammation and demyelination in Stat4α and Stat4β transgenic mice.

### Example 6

### Stat4β Transgenic Mice with EAE Express Elevated Levels of Effector T Cell-Derived Inflammatory Cytokines in the CNS and Lymphoid Organs

The mechanism in the differential regulation of EAE in Stat4α and Stat4β transgenic mice was analyzed. As mice that are deficient in Stat4 have multiple defects in Th1 differentiation, Th17 function, migration and adhesion of T cells to inflamed sites, the analysis was focused on comparing the Stat4α and Stat4β transgenic immune cells where differences likely reflect specific effects of the isoforms. Th1 differentiation in vitro is largely similar in Stat4α and Stat4β transgenic cells. The expression of effector T cell-derived inflammatory cytokines in the CNS, spleen and spleen cells cultured with antigen was measured. The levels of IFNγ and IL-17 mRNA detected in the brain and spleen of mice with EAE were significantly increased over unimmunized naïve mice and largely correlated to disease severity with tissues from wild type or Stat4β transgenic mice having the highest levels (FIG. 3). Expression of the Th1 transcription factor T-bet also correlated with IFNγ expression in tissues from Stat4β transgenic mice though less well in tissues from wild type mice. The mRNA levels of cytokines from antigen stimulated spleen cells were somewhat different with higher levels of IFNγ observed in wild type and Stat4β transgenic cultures but higher IL-17 in Stat4α transgenic cultures (FIG. 3). Thus, while the expression of IFNγ and IL-17 in the CNS correlated with disease severity, differences in cytokine profile between Stat4α and Stat4β transgenic mice indicate that Stat4 isoforms may differentially regulate cytokine production in EAE.

### Example 7

### Stat4β Transgenic Mice with EAE Express Elevated Levels of APC-Derived Inflammatory Cytokines in the CNS and Lymphoid Organs

To further define the mechanism in the differential regulation of EAE in Stat4α and Stat4β transgenic mice, the expression of antigen presenting cell-derived inflammatory cytokines in the CNS and lymphoid organs was examined. As shown in FIG. 4, the wild type and Stat4β transgenic mice with EAE showed an increased expression of IL-12p35, IL-12p40 and IL-23p19 in the brain and spleen compared to naïve mice. Stat4α transgenic mice with EAE showed little or no increase in the expression of IL-12p35, IL-12p40 or IL-23p19 in the brain and spleen. Interestingly, the levels of IL-12p35 mRNA correlated well with IFNγ mRNA levels and the levels of IL-12p40 and IL-23p19 mRNA correlated with IL-17 mRNA levels in both brain and spleen (compare FIG. 4 to FIG. 3) and with the clinical and pathological symptoms of EAE in Stat4α and Stat4β transgenic mice.

### Example 8

### IFNγ Production in the Periphery Correlates with the Severity of EAE in Stat4β Transgenic Mice

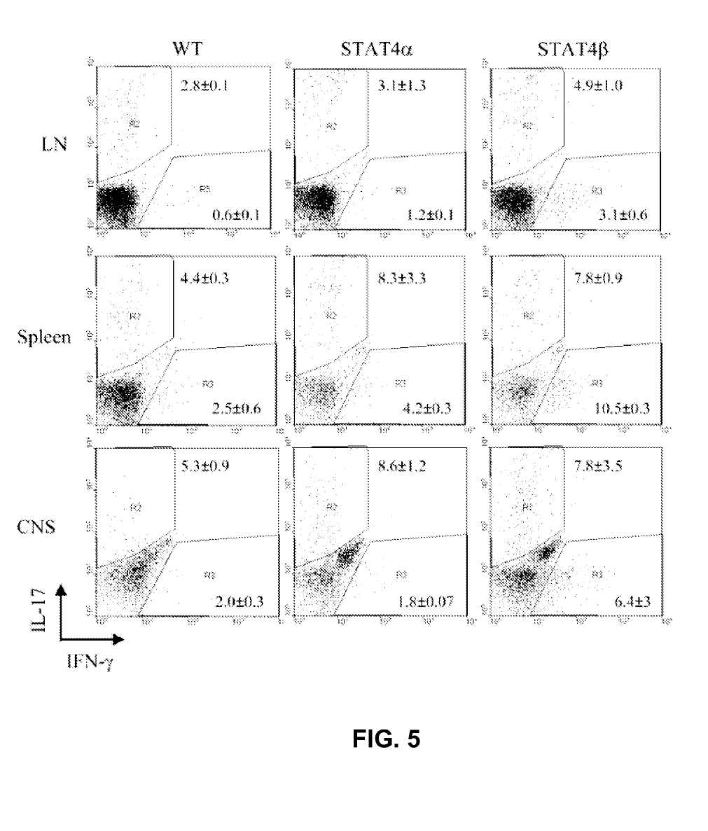

To determine if the differences observed in mRNA expression in FIGS. 3-4 results in differential cytokine production, IFNγ and IL-17 production by intracellular cytokine staining (FIG. 5) and ELISA (FIG. 6) were examined. Cells isolated from CNS, spleen or draining LN were stimulated with PMA and ionomycin before intracellular staining with anti-IL-17 and anti IFNγ antibodies in CD4+ cells. Early cytokine production in WT, Stat4α and Stat4β transgenic cells were not substantially different, with Stat4β transgenic cells having a slightly greater propensity for IFNγ production (FIG. 5). Moreover, while there was decreased inflammation in the Stat4α transgenic CNS (FIG. 2), Stat4α transgenic T cells in the CNS were capable of producing IL-17 and IFNγ at levels similar to wild-type cells (FIG. 5). In response to antigen stimulation, spleen cells from wild type and Stat4β transgenic mice produced higher levels of IFNγ than Stat4α transgenic cells, while, Stat4α transgenic cells produced more IL-17 than either wild type or Stat4β transgenic cells (FIG. 6). IL-12 or IL-23 production from antigen-stimulated spleen cells was not detected. These results highlight that the decreased disease in Stat4α transgenic mice is not due to an inability to develop inflammatory cell types in vivo.

To identify other genes that demonstrate Stat4-dependence, a Stat4 ChIP-on-chip experiment was performed. IL-10 was identified in this analysis and bound by Stat4 in the second and third introns (FIG. 7A). Wild type and Stat4−/− Th1 culture stimulated with either IL-12 or anti-CD3 demonstrated Stat4-dependence in the induction of IL10 production (FIG. 7B). As IL-10 is an important regulatory cytokine that inhibits the development of EAE, the regulation of IL-10 was examined by Stat4 isoforms. Wild type, Stat4α transgenic and Stat4β transgenic cells re-stimulated with anti-CD3 after culture under Th1 conditions demonstrated similar production of IFNγ (FIG. 7C). In contrast, Stat4β transgenic cells had decreased IL-10 production in these cultures (FIG. 7C). To test if this phenotype was reflected in vivo during disease, RNA from wild type, Stat4α transgenic and Stat4β transgenic mice were tested for IL10 expression in situ. Three-five fold more I110 mRNA was detected in Stat4α transgenic samples than in wild type or Stat4β transgenic samples (FIG. 7D). Splenic cultures from wild type, Stat4α transgenic and Stat4β transgenic were examined to assay for IL-10 production following stimulation as in FIG. 6. While wild type and Stat4β transgenic cells had similar IL-10 production, Stat4α transgenic cells produced 2-3-fold higher levels of IL-10 (FIG. 7E). Thus, Stat4α-expressing T cells have an increased propensity for IL-10 production and this is associated with decreased CNS inflammation and pathology in Stat4α transgenic mice, compared to wild type or Stat4β transgenic mice.

### Example 9

### Th1 Cells Expressing Stat4β Secrete Significantly More TNF-α Upon TCR Stimulation than Stat4α Expressing Th1 Cells

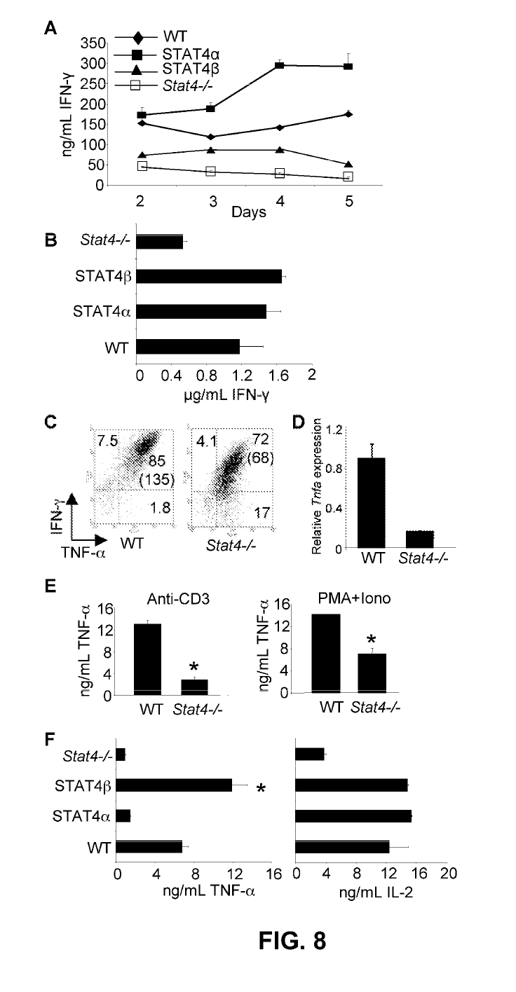

T cells expressing either Stat4α or STAT4β could differentiate into Th1 cells, Stat4α was more efficient than STAT4β in the induction of IFN-γ following IL-12 stimulation. Supernatants from naïve CD4+ T-cells undergoing Th1 differentiation in the presence of IL-12 for IFN-γ production (FIG. 8A) were examined. There was significantly less IFN-γ present in the supernatant throughout the differentiation period in Stat4β-expressing and Stat4-deficient cultures. Despite these differences, upon antiCD3 stimulation of differentiated Th1 cells, there were no significant differences in IFNY production between the isoforms (FIG. 8B). These results indicate that the differences in endogenous IFN-γ production stimulated by the Stat4 isoforms during the differentiation period, did not affect the process of differentiation.

Although IFN-γ levels were not different between Stat4α- and Stat4β-expressing Th1 cells, the levels of other cytokines were examined. The dependence of TNF-α production on Stat4 either in vitro or in vivo during the development of disease is not clear. To examine Stat4-dependent TNF-a production, wild type and Stat4-/naïve CD4+ T-cells were cultured in Th1 priming conditions for five days. At the end of the five-day culture, the cells were stimulated with IL-12, IL-12+IL-18, anti-CD3 or PMA+Ionomycin and analyzed for TNF-a and IFN-γ production. Maximal TNF-α production, as assessed by intracellular cytokine staining and mRNA levels, was dependent upon STAT4 (FIGS. 8C and D). While the percentage of TNF-a positive CD4+ T-cells did not differ drastically between wild type and Stat4−/− cells, the mean fluorescence intensity (MFI) at 4 hours and the secretion of TNF-a over a 24-hour time period showed TNF-a production significantly reduced in the absence of Stat4 (FIGS. 8C and E). In contrast, TNF-α production was not detected following stimulation with IL-12, in the presence or absence of IL-18.

Having demonstrated the STAT4-dependence in TNF-α production, the ability of the STAT4 isoforms to prime Th1 cells to secrete TNF-α was examined. Naïve CD4+ T-cells expressing either STAT4α or STAT4β were cultured under Th1 culture conditions for five days and stimulated with anti-CD3 before examining the levels of TNF-α and IL-2 using ELISA. The Th1 cells expressing STAT4β consistently secreted significantly more TNF-α compared to the CD4+ T-cells expressing STAT4α while IL-2 levels between cells expressing the STAT4 isoforms were similar (FIG. 8E). Similar to the data for Stat4−/− cells, decreased TNF-a production from STAT4α-expressing Th1 cells was due to decreased TNF-α per cell compared to STAT4β cultures, with only minor differences in the percentage of TNF-α+ cells, as assessed by intracellular cytokine staining These results indicate that IL-12 stimulation of STAT4β differentially programs the developing Th1 cells to secrete more TNF-α and that this programming is specific and independent of the concentration of IFN-γ throughout the culture period. Thus, these data indicate that STAT4 isoforms dictate differential cytokine expression in Th1 cells.

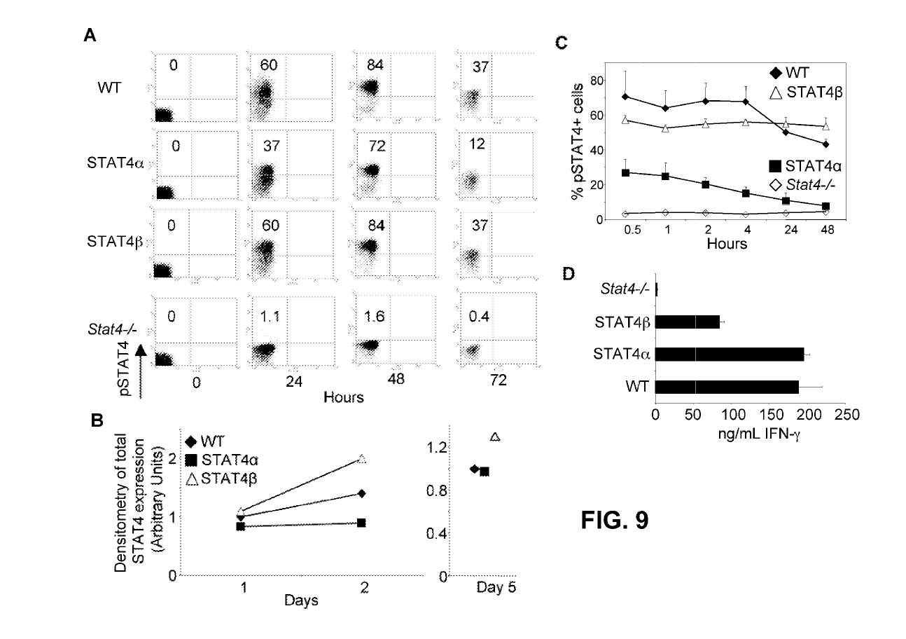

To determine if differential activation of STAT4 contributed to the production of distinct Th cytokines, developing Th1 cultures were stained for phospho-STAT4 (pSTAT4) levels over the first three days of culture. Wild type and STAT4β-expressing cells showed similar percentages of pSTAT4+ cells at all of the time points examined (FIG. 9A). In contrast, there was less pSTAT4 in STAT4α transgenic cells than in wild type cells or STAT4″ transgenic cells at all of the time points (FIG. 9A). During this time period there were modest changes in the expression of total STAT4 in each of the cell types (FIG. 9B). After five days of differentiation, IL-12 stimulation resulted in greater induction of pSTAT4 in wild type and STAT4β-expressing cells than in STAT4α-expressing cells, despite similar levels of total STAT4 expression (FIGS. 9B and C). Moreover, STAT4 expression did not change over the course of the stimulation. STAT4α phosphorylation decreased over time while STAT4β phosphorylation stayed constant over the 48-hour assay period (FIG. 9C). Despite lower levels of pSTAT4α during Th1 differentiation and following IL-12 restimulation, STAT4α was still more potent than STAT4β in the acute production of IFN-γ (FIG. 9D). These data indicate that the differential activation of the isoforms in response to IL-12 can contribute to differential gene expression but that the amount of activated STAT4 does not directly correlate with IFN-γ gene transcription.

### Example 10

### STAT4 Isoforms are Equally Efficient in Promoting Th17 Differentiation

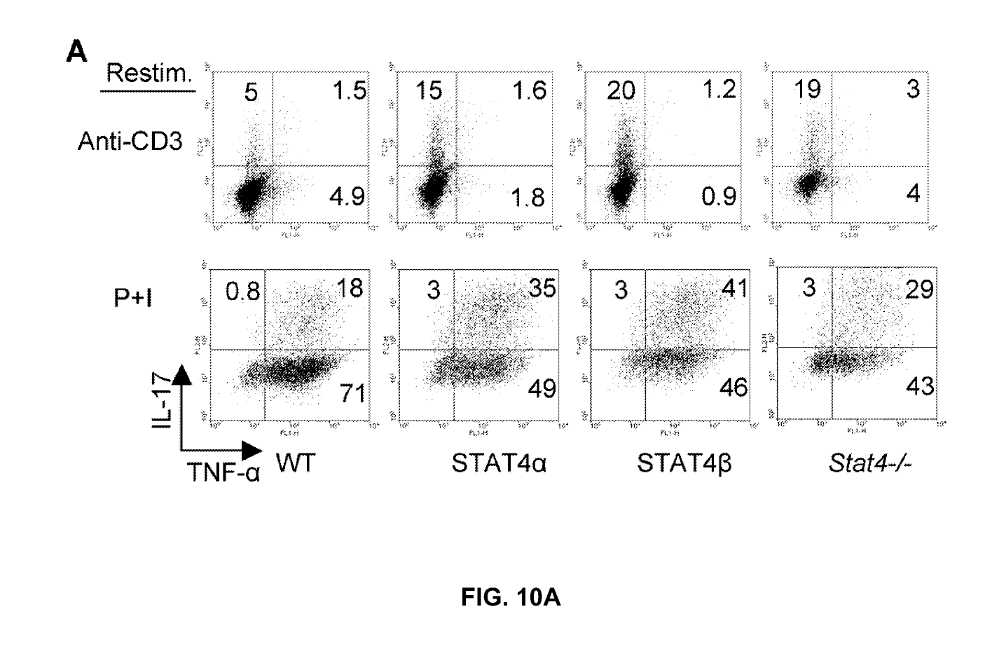

IL-23 also activates STAT4 and induces Th17 cells to secrete IL-17. The ability of Th17 cells expressing STAT4 isoforms to secrete IL-17 and TNF-α was examined. Naïve T-cells were differentiated with TGF-31, IL-6, and IL-23 for five days and stimulated cells with anti-CD3 or PMA+Ionomycin (FIG. 10A). There were no significant differences between the percentage of TNF-a positive cells in Th17 cells expressing either isoform although the percentage of TNF-a positive cells was considerably higher following PMA+ionomycin stimulation, compared to anti-CD3 (FIG. 10A). The Th17 cells expressing either isoform had similar capabilities to produce IL-17. Because generation of Th17 cells by TGFP+IL-6 is independent of STAT4, the effects of culture with IL-23 on IL-17 production from STAT4 isoform expressing T cells were examined. After a week of culture in IL-23 cells were restimulated with anti-CD3 and IL-17 production was analyzed using ELISA. There was also no defect in IL-17 production from T cells expressing either STAT4 isoform, and production was increased compared to wild type cells (FIG. 10B). To assess the responsiveness of the STAT4 isoforms to IL-23-induced cytokine production, IL-17 levels were examined by ELISA after 24 hours of stimulating the cells with IL-23 and IL-18 (FIG. 10C). T cells expressing the STAT4α isoform secreted similar amounts to wild type cells and significantly more IL-17 than cells expressing the STAT4β isoform. Thus, while either STAT4 isoform is sufficient for the generation of Th17 cells, activation of STAT4α by IL-23 can more efficiently induce IL-17 than the STAT4β isoform.

### Example 11

### STAT4β Promotes More Severe Colitic Inflammation than STAT4α

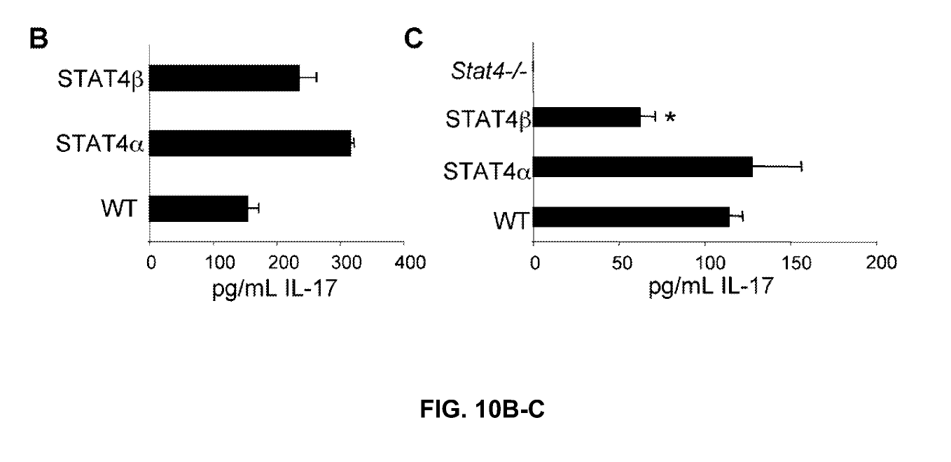

Since some differences were observed in the ability of T cells expressing STAT4α or STAT4β to secrete inflammatory cytokines, the ability of the T-cells expressing each isoform was tested to mediate inflammation. Therefore, SCID mice were reconstituted with CD4+CD45RBhigh or CD4+CD45RBlow T-cells that expressed either STAT4α or STAT4β and examined the weight loss kinetics of the mice. There was no significant difference in the kinetics of weight loss or the end point weight loss between the SCID mice reconstituted with either isoform or wild type mice (FIG. 11A). However, there was a significant difference between the weight loss of mice reconstituted with the CD4+CD45RBhigh cells compared to the mice reconstituted with CD4+CD45RBlow cells, indicating that the CD4+CD45RBhigh T-cells expressing either isoform were sufficient to induce colitis (FIG. 11A). As wild type mice had the same overall disease course as STAT4 isoform-expressing cells, the comparison between cells expressing the transgenic STAT4 isoforms was analyzed. To determine if the differences in T cell proliferation between the STAT4 isoforms resulted in differences in cell reconstitution in vivo, the absolute CD4+ cell numbers were determined in MLN cells and the percentage of CD4+ T-cells in the splenocytes and observed no significant difference between the repopulation efficiency of the CD4+ T-cells expressing either isoform (FIG. 11B). Similar to protein levels seen in FIG. 9B, STAT4 mRNA expression was slightly higher in STAT4β-expressing cells than STAT4α-expressing cells in vivo (FIG. 11B).

Although weight loss was not significantly different between the SCID mice reconstituted with either STAT4 isoform, gross examination of the colon and scoring of the slides showed that the SCID mice reconstituted with the CD4+CD45RBhigh cells expressing the STAT4β isoform had more significant mucosal inflammation than the SCID mice reconstituted with the STAT4α as assessed by area and severity of the lesion (FIG. 4C-E). There was no difference in mucosal hyperplasia between the mice reconstituted with STAT4α or STAT4β expressing T cells. Importantly, SCID mice reconstituted with the CD4+CD45RBlow cells had essentially no inflammatory infiltrates into the tissues (FIG. 11E).

### Example 12

### STAT4β Expressing T-Cells from Colitic Mice have Increased Inflammatory Cytokine Production Compared to Mice Reconstituted with STAT4α T-Cells

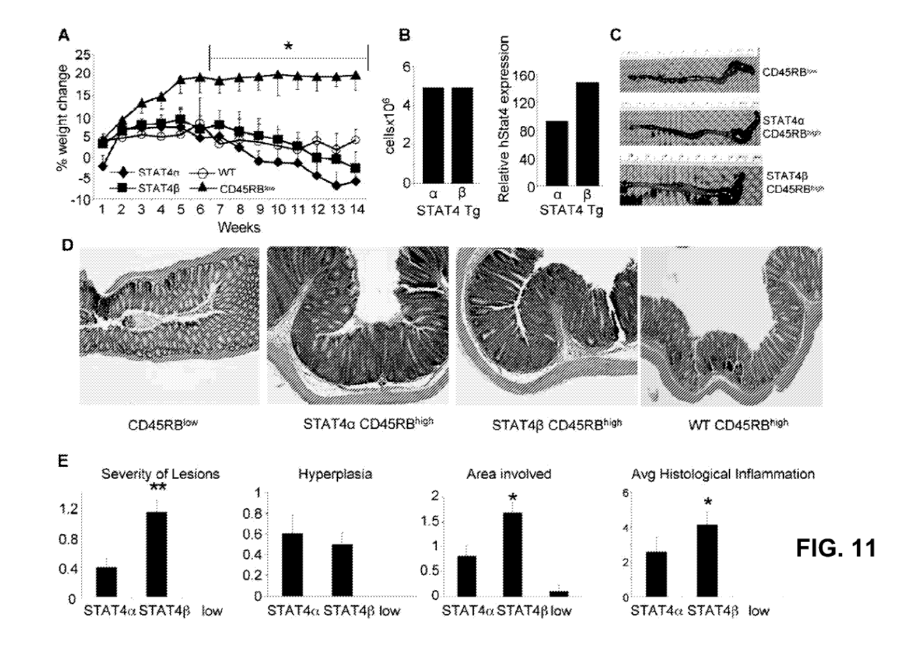

To examine whether the increased histological inflammation seen in the SCID mice reconstituted with the STAT4β expressing T-cells correlated with increased TNF-a production, isolated splenocytes and MLN cells were stimulated with anti-CD3 to assess ex vivo TNF-a production (FIG. 12A). The SCID mice reconstituted with the STAT4β expressing T-cells had significantly more TNF-a compared to the mice adoptively transferred with the STAT4α T-cells upon stimulation with anti-CD3. SCID mice reconstituted with CD4+CD45RBlow from either isoform had barely detectable TNF-α that was significantly less than the cells isolated from the SCID mice reconstituted with the CD45RBhigh subset of cells.

To determine if the STAT4 isoforms differentially regulated other cytokines in vivo, T-cell produced cytokines were examined that have been implicated in the pathogenesis of colitis, including IFN-γ, IL-6, IL-10, and IL-17 (FIG. 12B). Corresponding to the level of inflammation, SCID mice reconstituted with the STAT4β expressing T-cells had more inflammatory cytokine production. IFN-γ production was significantly increased from STAT4β expressing cells compared to STAT4α expressing cells from either spleen or MLNs. IL-6 production in the spleen was also increased but not in the MLNs of mice reconstituted with the STAT4β T-cells. IL-17 did not significantly differ between SCID mice reconstituted with T-cells expressing either isoform, and IL-10 was detected at higher levels from MLNs in mice reconstituted with STAT4α-expressing T cells but there was no significant difference in production detected from spleen cells (FIG. 12B). There was no significant difference in TGFP1 expression in MLN.

Since data in FIGS. 2-3 show that STAT4α is more efficient than STAT4β in cytokine stimulated production of IFN-γ and IL-17, the MLN cells from colitic mice were examined for their ability to produce these cytokines following treatment with IL-12 and IL-18 or IL-23 and IL-18 for 72 hours. While the IL-23 and IL18 stimulated cells from the SCID mice reconstituted with STAT4α secreted more IL-17, similar to results from in vitro differentiated cells, there was no significant difference in the amount of IFN-γ secreted from the cells isolated from the SCID mice reconstituted with either isoform (FIG. 12C). Overall, these data indicate that the increased inflammatory disease caused by STAT4β-expressing T cells correlates with increased inflammatory cytokine production.

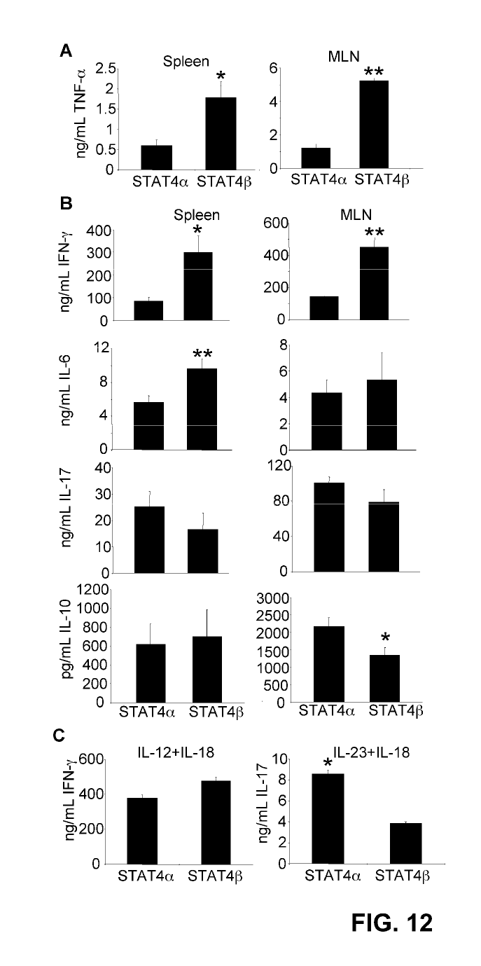

TNF-α and GM-CSF are important in neutrophil chemotaxis to inflamed tissues. To examine whether the increased TNF-α secretion from STAT4β expressing T-cells correlated with increased neutrophils in the lamina propria, microscopic sections of the colon for PMN infiltration were examined. Consistent with the increased TNF-a seen in the SCID mice reconstituted with the STAT4β isoform, there were also increased neutrophils present in the lamina propria compared to the SCID mice reconstituted with STAT4α (FIG. 13A). Since anti-TNF therapies have been shown to inhibit GM-CSF production, GM-CSF levels in the mice with colitis were analyzed. Supernatants from stimulated MLN cell cultures were examined to assess GM-CSF production (FIG. 13B). Consistent with the increased neutrophil infiltration, GM-CSF was significantly increased from STAT4β-expressing T cells, further supporting the ability of T-cells expressing the STAT4β isoform to mediate potent inflammatory responses.

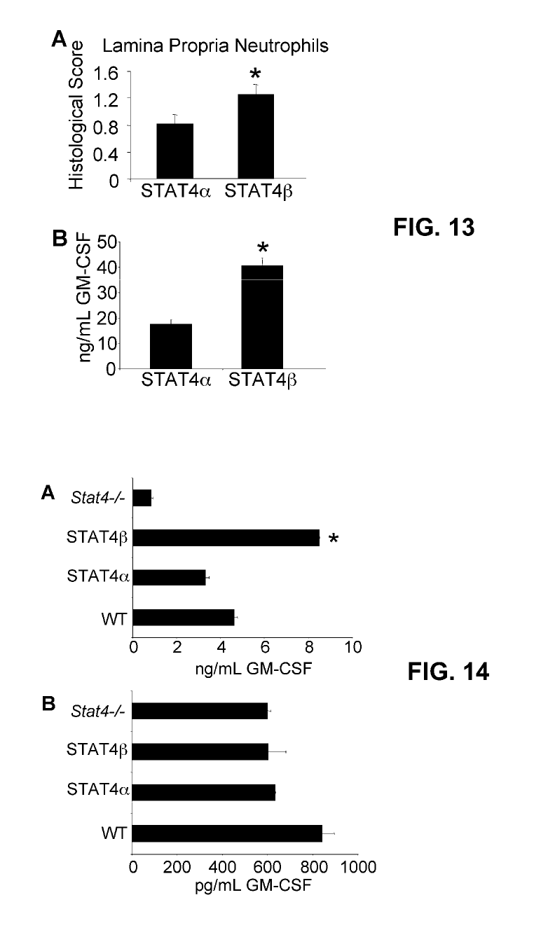

Since there was increased GM-CSF production from STAT4β-expressing cells ex vivo, it was examined whether this reflected an increased propensity for STAT4β expressing T cells to produce GM-CSF or whether it was a result of the in vivo inflammatory environment. To test this, naïve T-cells were isolated expressing either isoform and differentiated them in Th1 or Th17 conditions for five days and stimulated them with anti-CD3 to examine their ability to secrete GM-CSF. Production of GM-CSF in Th1 cultures was dependent upon STAT4 (FIG. 14a). The STAT4β expressing Th1 cells secreted significantly more GM-CSF than STAT4α expressing Th1 cells. In contrast, there was no STAT4 dependence for GM-CSF production from Th17 cells and no significant difference in the amount of GM-CSF produced by Th17 cells expressing either Stat4 isoform (FIG. 14B). No detectable GM-CSF was secreted upon acute stimulation with IL-12 or IL-23 with or without IL-18 suggesting that STAT4 does not directly induce transcription of GM-CSF. Together, these data demonstrate the increased inflammatory propensity of T cells expressing STAT4β and suggest that the increased inflammatory cytokine production by STAT4β-expressing T cells results in greater inflammatory disease in vivo.

### Materials and Methods

Animals: The C57BL/6 mice were purchased from Harlan (Indianapolis, Ind.). The Stat4−/− mouse in C57BL/6 background was generated as described earlier (26, 30). The transgenic mice expressing Stat4α or Stat4β genes were generated as described earlier (26). The mice were maintained in the animal care facility at Methodist Research Institute. All animal protocols used in the experiments were approved by the Institutional Animal Care and Use Committee.

Reagents: The 21 amino acid peptide [MEVGWYRSPFSRVVHLYRNGK] (SEQ ID NO: 9) corresponding to mouse MOGp35-55 was obtained from Genemed Synthesis. Inc. (San Francisco, Calif.). Murine recombinant IL-17, IFNγ and IL-10 were purchased from R&D Systems, Inc. (Minneapolis, Minn.). The biotin/FITC-conjugated anti-IL-17, anti-IFNγ and anti-IL-10 antibodies were purchased from e-Bioscience. All reagents for qRT-PCR were purchased from Applied Biosystems (Foster City, Calif.).

Induction of EAE: To induce EAE, 4 to 6 weeks old female mice (5 per group) were immunized (s.c.) with 100 μg of MOGp35-55 peptide antigen in 150 μl emulsion of Incomplete Freund's Adjuvant containing 50 μg/ml heat-killed Mycobacterium tuberculosis (H37Ra, Difco Laboratories, Detroit, Mich.) in the lower dorsum on days 0 and 7. The mice also received (i.p) 100 ng of pertussis toxin (Sigma Chemicals, St Louis, Mo.) on days 0 and 2. The clinical symptoms were scored every day from day 0 to 30 in a blinded manner as follows; 0, normal; 0.5, stiff tail; 1, limp tail; 1.5, limp tail with inability to right; 2, paralysis of one limb; 2.5, paralysis of one limb and weakness of one other limb; 3, complete paralysis of both hind limbs; 4, moribund; 5, death. Mean clinical score (MCS) was calculated by adding every day clinical score for all mice in a group and then divided by total number of mice. Mean maximum clinical score (MMCS) was the MCS at the peak of disease. Average mean clinical score (AMCS) was calculated by adding the MCS for all days (from day 0 to 30) and then divided by number of days. The mean clinical score more than one (MCS>1) was obtained by counting the number of days with MCS more than one. The area under the curve (AUC) was calculated using GraphPad Prism 5.0 Software.

Histological analysis: The mice induced to develop EAE were euthanized on day 30 by CO2 asphyxiation and perfused by intracardiac infusion of 4% paraformaldehyde and 1% glutaraldehyde in PBS. Brain and spinal cord samples were removed and fixed in 10% formalin in PBS. Tissues were processed and transverse sections from cervical, upper thoracic, lower thoracic and lumbar regions of the spinal cord were stained with Luxol Fast Blue or hematoxylin and eosin. Inflammation and demyelination in the CNS were assessed under microscope in a blinded manner. The spinal cord sections were viewed as anterior, posterior and two lateral columns (4 quadrants). Each quadrant displaying the infiltration of mononuclear cells or loss of myelin was assigned a score of one inflammation or one demyelination, respectively. Thus, each animal has a potential maximum score of 16 and this study represents the analysis of spinal cords from 5 mice per group. The pathological score from each group is expressed as percent positive over total number of quadrants examined.

Quantitative real-time polymerase chain reaction: The quantitative real-time reverse transcription polymerase chain reaction (qRT-PCR) was performed using the ABI Prism 7900 Fast Sequence Detection System (Applied Biosystems, Foster City, Calif.) according to manufacturer's instructions. The brain and spleen samples were isolated on day 14 following induction of EAE. Spleen cells were cultured in 24 well tissue culture plates in RPMI medium with 10 μg/ml MOGp35-55 antigen or 5 μg/ml Con A for 24 hrs. Total RNA was extracted from brain, spleen, and cultured spleen cells using TRIzol reagent (Invitrogen, Carlsbad, Calif.) according to manufacturer's instructions. The RNA samples (5 μg/100 μl reaction) were reverse transcribed into cDNA (RT-PCR) using random hexamer primers and TaqMan reverse transcription kit (Applied Biosystems, Foster City, Calif.). The cDNA (2 μl/sample) was subjected to qPCR analysis in triplicate using forward and reverse primers, TaqMan Universal Master Mix and probe (10 μl/reaction) in fast optical 96-well plate. Controls include RT-PCR in the absence of RNA and real-time PCR in the absence of cDNA. The data were analyzed using the ABI Prism 7900 relative quantification (delta-delta-Ct) study software (Applied Biosystems, Foster City, Calif.). In this study primer sets for 10 selected inflammatory genes were used with GAPDH (Applied Biosystems, Foster City, Calif.) as internal controls. The expression levels of inflammatory genes normalized to GAPDH are presented as arbitrary fold changes compared to control samples.

T cell proliferation assay: T cell proliferation was measured by WST-1 assay (Roche, Indianapolis, Ind.). The spleen cells were isolated on day 14 following induction of EAE and cultured in 96 well tissue culture plates in RPMI medium (1×10⁵/200 μl/well) with 0, 1, 5 and 10 μg/ml MOGp35-55 peptide. WST-1 reagent (10 μl/well) was added after 72 hrs of culture and the absorbance was measured at 450 nm using 2100 microplate reader (Alpha Diagnostics Inc., San Antonio, Tex.) as a measure of viable cell count.

Intracellular cytokine staining: Spleen, lymph node and brain cells isolated on day 14 following induction of EAE were cultured in 24 well tissue culture plates in RPMI medium (1×10⁶/ml) in the presence of 10 μg/ml MOGp35-55 antigen or 5 μg/ml Con A for 24 hrs. Monensin (2 μM) was added during the last 4 hrs to block protein secretion. The cells were isolated fixed and permeabilized by incubating in PBS containing 1% paraformaldehyde and 0.02% Triton X-100 at 4° C. for 15 min. After washing in PBS, the cells were stained with fluorochrome conjugated IL-17 and IFNγ antibodies at 4° C. for 30 min and analyzed using a three color FACSCanto flow cytometer to determine the percentage of cells expressing cytokines.

ELISA for IFNγ, IL-17 and IL-10: To determine the cytokine response, spleen cells from MOGp35-55-sensitized mice were cultured in 24 well plates in RPMI medium (1×10⁶/ml) in the presence of 0 and 10 μg/ml MOGp35-55 or 5 μg/ml Con A. The culture supernatants were collected after 48 hrs and the levels of IFNγ, IL-17 and IL-10 measured by ELISA. Briefly, 96-well ELISA plates were coated with 2 μg/ml of anti-IL-17 or anti-IFNγ capture antibody in 100 μl/well of 0.06 M Carbonate buffer, pH 9.6. After overnight incubation at 4° C., the excess Abs were washed off and the residual binding sites blocked by the addition of 100 μl/well of 1% BSA in PBS for 1 h. The test samples (culture supernatants) and standards (rIL-17, rIFNγ, rIL-10) were added and incubated at 4° C. overnight. Plates were washed with PBS containing 0.05% Tween-20 and 0.2 μg/ml of biotin-conjugated anti-IL-17, anti-IFNγ or anti-IL-10 added as detection antibody. After incubation at room temperature for 1 h, the plates were washed three times and avidin-alkaline phosphatase added followed by 1 mg/ml of p-nitrophenyl phosphate. After 30 min incubation at room temperature, the absorbance was read at 405 nm and the concentrations of IL-17, IFNγ and IL-10 in the culture supernatants were calculated from the standard curve. For some experiments, CD4+ T cells were cultured under Th1 conditions.

Statistical analysis: All the experiments were repeated two or three times and the values are expressed as mean±SD. The differences between groups were analyzed by ANOVA and the values of p<0.05 were considered significant.

A two sample Wilcoxon Rank Sum test is used to assess if patient samples with active Crohn's Disease, and/or ulcerative colitis have a higher α/β ratio than controls who are healthy. To evaluate the change in the α/β ratio at e.g., two weeks post treatment from baseline in response to e.g., corticosteroids or Infliximab treatment, Wilcoxon Signed Rank test is performed. Graphical presentation is also be used to show the patient specific change in α/β ratio. The point estimate and 95% confidence interval for the mean of the α/β ratio is reported on the log scale by group.

Isolation of CD45RBhi and CD45RBlowCD4+ T-cells and Induction of Colitis by Cell Transfer: Spleen and lymph node cells were used as a source of CD4+ cells for reconstitution of B6 SCID recipient mice. CD4+ T-cells were isolated as previously described (28). The enriched CD4+ T-cells were then labeled for cell sorting with FITC-conjugated CD4 and PE-conjugated CD45RB (BD Pharmingen). Subsequently, cells were sorted under sterile conditions by flow cytometry for CD4+CD45RBhi on a FacsVantage machine (Becton Dickinson). The CD45RBhigh and CD45RBlow populations were defined as the brightest staining 10-15% and the dullest staining 15-20% CD4+ T cells, respectively. Intermediate staining populations were discarded. All populations were >99% pure on re-analysis. The purified CD45RBhiCD4+ (4×10⁵) cells diluted in 200 μl of PBS were injected intraperitoneally into B6 SCID recipient mice. A separate group of B6 SCID mice received CD45RBlowCD4+ (4×10⁵) cells as a negative control. The recipient mice were weighed initially, then weekly thereafter. The animals were sacrificed 14 weeks after transfer.

Macroscopic and microscopic assessment of Colon Appearance: Once the animals were sacrificed, tissue samples were taken from each segment of the colon (cecum, ascending, transverse, and descending colon and rectum) and fixed in 10% neutral buffered formalin. The samples were routinely processed, sectioned at 5 μm thickness, and stained with hematoxylin and eosin (H&E) for light microscopic examination. The slides were evaluated by light microscopy in a blind fashion using a semi-quantitative scoring system. In brief, four general criteria were evaluated in all sections: (1) severity, (2) degree of mucosal hyperplasia, (3) degree of ulceration, if present, and (4) percentage of area involved. The score was then determined from each slide by the following mathematical formula: ((inflammation score+ulceration score+hyperplasia score)×(Area involved score)) for a score range of 0-27. Scores from each section of the colon were averaged to determine the overall histological score per experimental group. Histological grades were assigned in a blinded fashion. For scoring the lamina propria neutrophils, the following scoring system was used and scores were averaged from 5-10 high-powered fields: 0-0-5 PMNs, 1-6-10 PMNs, 2-11-20 PMNs, 3-21 PMNs and above.

Blood samples: Peripheral blood samples is also obtained for IL-12, IL-23, IL-17 and TNF [symbol] levels, Sed rate (ESR) and Hematocrit. The pediatric Crohn's disease activity index, the Rachmilewitz Clinical Activity index and the Colitis Symptom Score is also assessed.

```
Stat4α isoform nucleotide sequence (2247 nt)
(SEQ ID NO: 1)
ATGTCTCAGTGGAATCAAGTCCAACAGTTAGAAATCAAGTTTTTGGAGCAGGTGGATCAATTCTATGATG
ACAACTTTCCCATGGAAATTCGGCATCTGTTGGCCCAATGGATTGAAAATCAAGACTGGGAGGCAGCTTC
TAACAATGAAACCATGGCAACGATTCTTCTTCAAAACTTGTTAATACAACTGGATGAACAGTTAGGTCGT
GTTTCCAAAGAGAAAAACCTACTCTTGATACACAATCTAAAAAGAATTAGGAAGGTCCTTCAGGGAAAAT
TTCATGGAAATCCAATGCATGTAGCTGTGGTTATTTCAAACTGTTTAAGGGAAGAGAGGAGAATATTGGC
TGCAGCCAACATGCCTGTCCAGGGGCCTCTAGAGAAATCCTTACAAAGTTCTTCAGTTTCAGAAAGACAG
AGGAATGTGGAGCACAAAGTGGCTGCCATTAAAAACAGTGTGCAGATGACAGAACAAGATACCAAATACT
TAGAAGATCTGCAAGACGAATTTGACTACAGGTATAAAACAATTCAGACAATGGATCAGAGTGACAAGAA
TAGTGCCATGGTGAATCAGGAAGTTTTGACACTGCAGGAAATGCTTAACAGCCTCGATTTCAAGAGAAAG
GAGGCTCTCAGTAAAATGACCCAAATCATCCATGAGACAGACCTGTTAATGAACACCATGCTCATAGAAG
AGCTGCAAGACTGGAAGCGGCGGCAGCAAATCGCCTGCATCGGGGGTCCACTCCACAATGGGCTCGACCA
GCTTCAGAACTGCTTTACACTATTGGCAGAAAGTCTTTTCCAACTGAGAAGGCAATTGGAGAAACTAGAG
GAGCAATCTACCAAAATGACATATGAAGGTGATCCCATTCCAATGCAAAGAACTCACATGCTAGAAAGAG
TCACCTTCTTGATCTACAACCTTTTCAAGAACTCATTTGTGGTTGAGCGACAGCCATGTATGCCAACCCA
CCCTCAGAGGCCGTTGGTACTTAAAACCCTAATTCAGTTCACTGTAAAACTAAGGCTACTAATAAAATTG
CCAGAACTAAACTATCAGGTAAAGGTTAAGGCATCAATTGACAAGAATGTTTCAACTCTAAGCAACCGAA
GATTTGTACTTTGTGGAACTAATGTCAAAGCCATGTCTATTGAAGAATCTTCCAATGGGAGTCTCTCAGT
AGAATTTCGACATTTGCAACCAAAGGAAATGAAGTCCAGTGCTGGAGGTAAAGGAAATGAGGGCTGTCAC
ATGGTGACTGAAGAACTTCATTCCATAACGTTTGAAACACAGATCTGCCTCTATGGCCTGACCATAGATT
TGGAGACCAGCTCATTGCCTGTGGTGATGATTTCCAATGTCAGTCAGTTACCTAATGCTTGGGCATCCAT
CATTTGGTACAACGTGTCAACCAACGATTCCCAGAACTTGGTTTTCTTTAATAATCCTCCACCTGCCACA
TTGAGTCAACTACTGGAGGTGATGAGCTGGCAGTTTTCATCGTACGTTGGTCGTGGTCTTAACTCAGATC
AACTCCATATGCTGGCAGAGAAGCTTACAGTCCAATCTAGCTACAGTGATGGTCACCTCACCTGGGCCAA
GTTCTGCAAGGAACATTTACCTGGTAAATCATTTACCTTTTGGACATGGCTTGAAGCAATATTGGATCTA
ATTAAGAAACACATTCTTCCCCTTTGGATTGATGGGTATGTCATGGGCTTTGTTAGCAAAGAGAAGGAAC
GGCTGTTGCTAAAGGATAAAATGCCTGGCACCTTTTTATTAAGATTCAGTGAAAGCCATCTCGGAGGAAT
AACTTTCACCTGGGTGGACCATTCTGAAAGTGGGGAAGTGAGATTCCACTCTGTAGAACCCTACAATAAA
GGCCGGTTGTCTGCTCTGCCATTCGCTGACATCCTGCGAGACTACAAAGTTATTATGGCTGAAAACATTC
CTGAAAACCCTCTGAAGTACCTATATCCTGACATTCCCAAAGACAAAGCCTTCGGTAAACACTACAGCTC
TCAGCCTTGCGAAGTTTCAAGACCAACAGAAAGGGGTGACAAAGGTTATGTTCCTTCTGTTTTTATCCCC
ATCTCAACAATCCGAAGTGATTCAACAGAGCCACATTCTCCATCAGACCTTCTTCCCATGTCTCCAAGTG
TGTATGCGGTGTTGAGAGAAAACCTGAGTCCCACAACAATTGAAACTGCAATGAAGTCTCCTTATTCTGC
TGAATGA
STAT4β Nucleotide Sequence
(lowercase represents the beta-specific exon sequence)
(SEQ ID NO: 2)
ATGTCTCAGTGGAATCAAGTCCAACAGTTAGAAATCAAGTTTTTGGAGCAGGTGGATCAATTCTATGATG
ACAACTTTCCCATGGAAATTCGGCATCTGTTGGCCCAATGGATTGAAAATCAAGACTGGGAGGCAGCTTC
TAACAATGAAACCATGGCAACGATTCTTCTTCAAAACTTGTTAATACAACTGGATGAACAGTTAGGTCGT
GTTTCCAAAGAGAAAAACCTACTCTTGATACACAATCTAAAAAGAATTAGGAAGGTCCTTCAGGGAAAAT
TTCATGGAAATCCAATGCATGTAGCTGTGGTTATTTCAAACTGTTTAAGGGAAGAGAGGAGAATATTGGC
TGCAGCCAACATGCCTGTCCAGGGGCCTCTAGAGAAATCCTTACAAAGTTCTTCAGTTTCAGAAAGACAG
AGGAATGTGGAGCACAAAGTGGCTGCCATTAAAAACAGTGTGCAGATGACAGAACAAGATACCAAATACT
TAGAAGATCTGCAAGACGAATTTGACTACAGGTATAAAACAATTCAGACAATGGATCAGAGTGACAAGAA
TAGTGCCATGGTGAATCAGGAAGTTTTGACACTGCAGGAAATGCTTAACAGCCTCGATTTCAAGAGAAAG
GAGGCTCTCAGTAAAATGACCCAAATCATCCATGAGACAGACCTGTTAATGAACACCATGCTCATAGAAG
AGCTGCAAGACTGGAAGCGGCGGCAGCAAATCGCCTGCATCGGGGGTCCACTCCACAATGGGCTCGACCA
GCTTCAGAACTGCTTTACACTATTGGCAGAAAGTCTTTTCCAACTGAGAAGGCAATTGGAGAAACTAGAG
GAGCAATCTACCAAAATGACATATGAAGGTGATCCCATTCCAATGCAAAGAACTCACATGCTAGAAAGAG
TCACCTTCTTGATCTACAACCTTTTCAAGAACTCATTTGTGGTTGAGCGACAGCCATGTATGCCAACCCA
CCCTCAGAGGCCGTTGGTACTTAAAACCCTAATTCAGTTCACTGTAAAACTAAGGCTACTAATAAAATTG
CCAGAACTAAACTATCAGGTAAAGGTTAAGGCATCAATTGACAAGAATGTTTCAACTCTAAGCAACCGAA
GATTTGTACTTTGTGGAACTAATGTCAAAGCCATGTCTATTGAAGAATCTTCCAATGGGAGTCTCTCAGT
AGAATTTCGACATTTGCAACCAAAGGAAATGAAGTCCAGTGCTGGAGGTAAAGGAAATGAGGGCTGTCAC
ATGGTGACTGAAGAACTTCATTCCATAACGTTTGAAACACAGATCTGCCTCTATGGCCTGACCATAGATT
TGGAGACCAGCTCATTGCCTGTGGTGATGATTTCCAATGTCAGTCAGTTACCTAATGCTTGGGCATCCAT
CATTTGGTACAACGTGTCAACCAACGATTCCCAGAACTTGGTTTTCTTTAATAATCCTCCACCTGCCACA
TTGAGTCAACTACTGGAGGTGATGAGCTGGCAGTTTTCATCGTACGTTGGTCGTGGTCTTAACTCAGATC
AACTCCATATGCTGGCAGAGAAGCTTACAGTCCAATCTAGCTACAGTGATGGTCACCTCACCTGGGCCAA
GTTCTGCAAGGAACATTTACCTGGTAAATCATTTACCTTTTGGACATGGCTTGAAGCAATATTGGATCTA
ATTAAGAAACACATTCTTCCCCTTTGGATTGATGGGTATGTCATGGGCTTTGTTAGCAAAGAGAAGGAAC
GGCTGTTGCTAAAGGATAAAATGCCTGGCACCTTTTTATTAAGATTCAGTGAAAGCCATCTCGGAGGAAT
AACTTTCACCTGGGTGGACCATTCTGAAAGTGGGGAAGTGAGATTCCACTCTGTAGAACCCTACAATAAA
GGCCGGTTGTCTGCTCTGCCATTCGCTGACATCCTGCGAGACTACAAAGTTATTATGGCTGAAAACATTC
CTGAAAACCCTCTGAAGTACCTATATCCTGACATTCCCAAAGACAAAGCCTTCGGTAAACACTACAGCTC
TCAGCCTTGCGAAGTTTCAAGACCAACAGAAAGGGGTGACAAAGGTTATGTTCCTTCTGTTTTTATCCCC
ATCTCAACAATgtgagtaatgttagtcacatgtgaaatatttttataaaaagctttcctataggagattt
aaaggtagagcagagtacacataactgagaacaaagcattgtaatgtgcaatgtcccatttcctttaata
cataaggctagccttcagggcacacttaccacaatctattgtgcctaaaattataaaattccccttttat
atgccatatatgccacagtaagttgagtgttctgatatgaaatgatgaattagataactcaatgtcacaa
atagatgaagccctagaaatgagttcctgacatagtaagtcaccgtgaactattattattttttaatcct
tgtccatattgaccttgttatctctttaagCCGAAGTGATTCAACAGAGCCACATTCTCCATCAGACCTT
CTTCCCATGTCTCCAAGTGTGTATGCGGTGTTGAGAGAAAACCTGAGTCCCACAACAATTGAAACTGCAA
TGAAGTCTCCTTATTCTGCTGAATGA
STAT413-specific exon sequence
(SEQ ID NO: 3)
gtgagtaatgttagtcacatgtgaaatatttttataaaaagctttcctataggagatttaaaggta
gagcagagtacacataactgagaacaaagcattgtaatgtgcaatgtcccatttcctttaatacat
aaggctagccttcagggcacacttaccacaatctattgtgcctaaaattataaaattcccctttta
tatgccatatatgccacagtaagttgagtgttctgatatgaaatgatgaattagataactcaatgt
cacaaatagatgaagccctagaaatgagttcctgacatagtaagtcaccgtgaactattattattt
tttaatccttgtccatattgaccttgttatctctttaag
STAT4α Amino Acid Sequence
(SEQ ID NO: 4)
MSQWNQVQQL EIKFLEQVDQ FYDDNFPMEI RHLLAQWIEN QDWEAASNNE TMATILLQNL
                70 80 90 100 110 120
LIQLDEQLGR VSKEKNLLLI HNLKRIRKVL QGKFHGNPMH VAVVISNCLR EERRILAAAN
              130 140 150 160 170 180
MPVQGPLEKS LQSSSVSERQ RNVEHKVAAI KNSVQMTEQD TKYLEDLQDE FDYRYKTIQT
              190 200 210 220 230 240
MDQSDKNSAM VNQEVLTLQE MLNSLDFKRK EALSKMTQII HETDLLMNTM LIEELQDWKR
              250 260 270 280 290 300
RQQIACIGGP LHNGLDQLQN CFTLLAESLF QLRRQLEKLE EQSTKMTYEG DPIPMQRTHM
              310 320 330 340 350 360
LERVTFLIYN LFKNSFVVER QPCMPTHPQR PLVLKTLIQF TVKLRLLIKL PELNYQVKVK
              370 380 390 400 410 420
ASIDKNVSTL SNRRFVLCGT NVKAMSIEES SNGSLSVEFR HLQPKEMKSS AGGKGNEGCH
              430 440 450 460 470 480
MVTEELHSIT FETQICLYGL TIDLETSSLP VVMISNVSQL PNAWASIIWY NVSTNDSQNL
              490 500 510 520 530 540
VFFNNPPPAT LSQLLEVMSW QFSSYVGRGL NSDQLHMLAE KLTVQSSYSD GHLTWAKFCK
              550 560 570 580 590 600
EHLPGKSFTF WTWLEAILDL IKKHILPLWI DGYVMGFVSK EKERLLLKDK MPGTFLLRFS
              610 620 630 640 650 660
ESHLGGITFT WVDHSESGEV RFHSVEPYNK GRLSALPFAD ILRDYKVIMA ENIPENPLKY
              670 680 690 700 710 720
LYPDIPKDKA FGKHYSSQPC EVSRPTERGD KGYVPSVFIP ISTIRSDSTE PHSPSDLLPM
              730 740
SPSVYAVLRE NLSPTTIETA MKSPYSAE
STAT4®-Amino Acid Sequence
(SEQ ID NO: 5)
MSQWNQVQQL EIKFLEQVDQ FYDDNFPMEI RHLLAQWIEN QDWEAASNNE TMATILLQNL
                70 80 90 100 110 120
LIQLDEQLGR VSKEKNLLLI HNLKRIRKVL QGKFHGNPMH VAVVISNCLR EERRILAAAN
              130 140 150 160 170 180
MPVQGPLEKS LQSSSVSERQ RNVEHKVAAI KNSVQMTEQD TKYLEDLQDE FDYRYKTIQT
              190 200 210 220 230 240
MDQSDKNSAM VNQEVLTLQE MLNSLDFKRK EALSKMTQII HETDLLMNTM LIEELQDWKR
              250 260 270 280 290 300
RQQIACIGGP LHNGLDQLQN CFTLLAESLF QLRRQLEKLE EQSTKMTYEG DPIPMQRTHM
              310 320 330 340 350 360
LERVTFLIYN LFKNSFVVER QPCMPTHPQR PLVLKTLIQF TVKLRLLIKL PELNYQVKVK
              370 380 390 400 410 420
ASIDKNVSTL SNRRFVLCGT NVKAMSIEES SNGSLSVEFR HLQPKEMKSS AGGKGNEGCH
              430 440 450 460 470 480
MVTEELHSIT FETQICLYGL TIDLETSSLP VVMISNVSQL PNAWASIIWY NVSTNDSQNL
              490 500 510 520 530 540
VFFNNPPPAT LSQLLEVMSW QFSSYVGRGL NSDQLHMLAE KLTVQSSYSD GHLTWAKFCK
              550 560 570 580 590 600
EHLPGKSFTF WTWLEAILDL IKKHILPLWI DGYVMGFVSK EKERLLLKDK MPGTFLLRFS
              610 620 630 640 650 660
ESHLGGITFT WVDHSESGEV RFHSVEPYNK GRLSALPFAD ILRDYKVIMA ENIPENPLKY
              670 680 690 700
LYPDIPKDKA FGKHYSSQPC EVSRPTERGD KGYVPSVFIP ISTM
```

1. A method of predicting the likelihood of successful anti-inflammatory therapy for a patient suffering from an inflammatory disease, the method comprising predicting the likelihood of successful anti-TNF therapy for the patient based on an expression ratio of Stat4β/Stat4α in a biological sample from the patient.

2. The method of claim 1, wherein the biological sample is a tissue biopsy or blood.

3. The method of claim 1, wherein the expression ratio is determined by analyzing the expression in isolated peripheral blood mononuclear cells (PBMC).

4. The method of claim 1, wherein the biological sample includes T-cells.

5. The method of claim 1, wherein the inflammatory disease or response is selected from the group consisting of Crohn's disease, ulcerative colitis, inflammatory bowel disease (IBD), rheumatoid arthritis, lupus, psoriasis, and multiple sclerosis (MS).

6. The method of claim 1, wherein the ratio of Stat4β/Stat4α ranges from about to 2 to 60.0.

7. The method of claim 1, wherein the ratio of Stat4β/Stat4α is greater than 10.

8. The method of claim 1, wherein the expression ratio is determined by a technique selected from the group consisting of PCR, quantitative PCR or real-time PCR, semi-quantitative PCR, probe-hybridization, mass spectrometry, and antibody-based quantitation.

9. The method of claim 1 further comprising evaluating clinicopathological data selected from the group consisting of patient age, previous personal and/or familial history of inflammatory diseases, previous personal and/or familial history of response to anti-inflammatory therapy, and presence of one or more single nucleotide polymorphisms (SNPs) associated with the Stat4 isoforms.

10. The method of claim 1, wherein the anti-inflammatory therapy is selected from the group consisting of infliximab, adalimumab, certolizumab pegol, afelimomab, golimumab, etanercept, abatacept, and anakinra.

11. The method of claim 1, wherein the expression ratio of Stat4β/Stat4α is applied for clinical detection of disease, disease diagnosis, disease prognosis, or treatment outcome or a combination of thereof relating to an inflammatory disorder.

12. The method of claim 1, wherein predicting the likelihood of successful anti-TNF therapy for the patient based on the Stat4β/Stat4α expression ratio includes correlating the ratio of Stat4β/Stat4α in the patient to a control sample or a reference value.

13. A method of treating an individual suffering from or suspected of suffering from an inflammatory disease, the method comprising:
(a) determining whether a sample from the individual has a higher Stat4β/Stat4α expression ratio as compared to a control or a reference value; and
(b) administering an anti-TNF therapy if the individual has a higher Stat4β/Stat4α ratio.

14. The method of claim 13, wherein the individual is suffering from the inflammatory disease selected from the group consisting of Crohn's disease and ulcerative colitis.

15. The method of claim 13, wherein the anti-TNF therapy is selected from the group consisting of infliximab, adalimumab, certolizumab pegol, afelimomab, golimumab, etanercept, abatacept, and anakinra.

16. A method of assessing a patient's risk for developing an inflammatory disease or an inflammatory response, the method comprising:
(a) quantifying the expression level of Stat4α and Stat4β isoforms in a biological sample from the patient; and
(b) determining that the patient's risk for the inflammatory disease or the inflammatory response is higher if the patient exhibits a higher Stat4β:Stat4α ratio as compared to a control.

17. A method of predicting disease severity in a patient's suspected of suffering from an inflammatory disease, the method comprising:
(a) obtaining the expression level of Stat4α and Stat4β isoforms in a biological sample from the patient; and
(b) determining that the disease severity for the patient suffering from the inflammatory disease is higher if the patient exhibits a higher Stat4β:Stat4α ratio as compared to a control.

18. A method of preventing or minimizing excessive inflammatory response in an immuno compromised patient, the method comprising:
(a) determining if the patient exhibits higher risk for the inflammatory response based on the patient's Stat4β:Stat4α expression level ratio in a biological sample as compared to a control; and
(b) administering an anti-inflammatory therapy to minimize the excessive inflammatory response.

19. The method of claim 18, wherein the excessive inflammatory response is associated with sepsis.

20. The method of claim 18, wherein the immune compromised patient is treated with an immuno suppressive agent.

21. A diagnostic kit to predict the response to anti-inflammatory therapy comprising reagents to specifically quantify the expression levels of Stat4α and Stat4β isoforms.

22. The diagnostic kit of claim 21, wherein the reagents are oligonucleotide primers that specifically amplify a portion of Stat4β and Stat4α isoforms.

23. The diagnostic kit of claim 21, wherein the reagents are oligonucleotide primers selected from the group consisting of 5′-TAT CCT GAC ATT CCC AAA GAC-3′ (SEQ ID NO: 6), 5′-CTC TCA ACA CCG CAT ACA CAC-3′ (SEQ ID NO: 7), and 5′ GAC TTA CTA TGT CAG GAA CTC-3′ (SEQ ID NO: 8).

24. The diagnostic kit of claim 21, wherein the reagents are Stat4β and Stat4α-specific antibodies.

25. The diagnostic kit of claim 21, wherein the reagents are nucleic acid probes that specifically hybridize to at least a portion of Stat4β and Stat4α isoforms.

26. The diagnostic kit of claim 25, wherein the probes bind to a region of Stat4β comprising SEQ ID NO: 5 under high stringency hybridization conditions.

27. A nucleic acid probe comprising a contiguous region of about 15 nucleotides of SEQ ID NO: 5, wherein the probe is capable of selectively binding to the Stat4β-specific exon.

28. The nucleic acid probe of claim 27, wherein the probe comprises a reverse complementary strand capable of selectively binding to SEQ ID NO: 5.

29. The nucleic acid probe of claim 27, wherein the probe consists essentially of a sequence of about 15-20 nucleotides capable of selectively binding to SEQ ID NO: 5.

30. A method of identifying an agent for modulating an inflammatory response, the method comprising:
(a) contacting a population of cells with a candidate agent; and
(b) identifying the candidate agent as the agent for modulating immunex response if the expression level of Stat4β isoform is reduced.

31. The method of claim 30, wherein the candidate agent is a small molecule.

32. The method of claim 30, wherein the inflammatory response is modulated in a disease selected from the group consisting of Crohn's disease, ulcerative colitis, rheumatoid arthritis, lupus, psoriasis, and multiple sclerosis.

33. The method of claim 30, wherein the expression level of Stat4β isoform is reduced without substantially reducing the expression level of Stat4α isoform.

34. The method of claim 30, wherein the expression level of Stat4β isoform is selectively reduced by an agent comprising a siRNA.
